(19) United States
(12) Patent Application Publication
(10) Pub. No.: US 2026/0260736 A1
(43) Pub. Date: Sep. 3, 2026

(54) SYSTEM, METHOD, AND APPARATUS FOR LABELING AND TRACKING LABORATORY SAMPLES

(71) Applicant: EMD Millipore Corporation, Burlington, MA (US)

(72) Inventors: Ryan Amara, Tewksbury, MA (US); Michael Miller, North Andover, MA (US); Lindsay D. Lozeau, Tyngsborough, MA (US)

(21) Appl. No.: 19/163,248

(22) PCT Filed: Mar. 6, 2024
(86) PCT No.: PCT/US2024/018615

§ 371 Date: Sep. 8, 2025

Related U.S. Application Data
(60) Provisional application No. 63/489,060, filed on Mar. 8, 2023.

(51) Int. Cl. G16H 40/20; G06K 19/06; G16H 10/40; G16H 70/20

(52) U.S. Cl. CPC G16H 40/20; G06K 19/06028; G16H 10/40; G16H 70/20

(57) ABSTRACT

A device and related method for laboratory workflow handling is disclosed. The device may include one or more processors configured to provide a plurality of actions for selection by a user. A user can select an action of the plurality of actions and the device can determine an identification corresponding to a sample. The device can embed within a two-dimensional barcode the identification and workflow data corresponding to the sample and print a label with the two-dimensional barcode on an adhesive material using a printer.

### BACKGROUND

### Relevant Field

The present disclosure relates to labeling and tracking of workpieces. More particularly, the present disclosure relates to a system, method and apparatus for labeling and tracking laboratory samples.

### Description of Related Art

Samples in a laboratory setting are labelled and tracked to maintain the integrity and traceability of the samples during various procedures, including reagent aliquoting, mixing, experimentation, dilution, and storing reagents in designated locations in proper environmental conditions. Accurate sample labelling can improve the reliability of experimental results.

Traditionally, labeling of laboratory samples has been done manually, using handwritten labels or pre-printed labels that are affixed to the samples. Manually tracking and managing these details can be time-consuming and prone to errors, especially as the number of samples increases. This method is labor-intensive and time consuming, which can negatively impact the efficiency of laboratory workflows.

To address this, various lab labeling systems have been developed to automate and streamline the process of managing, tracking, and storing samples. These systems typically involve the use of labels that are attached to the samples and contain information such as a unique identifier, the name of the sample, and any relevant details. These systems typically involve the use of specialized software and hardware to create, track, and print labels for samples, as well as to store and manage information about the samples. By automating the labeling process, these systems can help to reduce errors, improve efficiency, and enhance the traceability and integrity of samples. This is especially important in regulated industries such as pharmaceuticals and food production, where the traceability of samples is used to ensure product quality and safety.

Labelling systems can also improve the organization and management of samples within a laboratory. By providing a centralized, digital record of all samples and their relevant information, labelling systems can make it easier to locate and track samples, and to keep track of expiration dates and other important details. Thus, labelling systems can help to improve the efficiency, accuracy, and reliability of laboratory workflows by automating the labelling process and maintaining a comprehensive record of samples.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, a method may include providing a plurality of actions for selection by a user; selecting an action of the plurality of actions; determining an identification corresponding to a sample; and embedding within a two-dimensional barcode the identification and workflow data corresponding to the sample. The method may moreover include printing a label with the two-dimensional barcode on an adhesive material using a printer. Other embodiments of this aspect include corresponding computer systems, apparatus, and computer programs recorded on one or more computer storage devices, each configured to perform the actions of the methods. The workflow data may be a string in human-readable format. The workflow data may be experimental utilization of the sample such that the data is utilized to coordinate handling of the sample. The method may include a guided menu configured to guide the user to input the workflow data.

The selected action may be an aliquot action. The method may include: providing a create reagent label action and providing a create aliquot labels action. The method may include: selecting the create reagent label action; prompting the user with a request for a plurality of reagent parameters; receiving the plurality of reagent parameters; and embedding within the two-dimensional barcode the plurality of reagent parameters corresponding to the sample. The method may include storing the plurality of reagent parameters and the identification corresponding to the sample in a database.

The method may include: selecting an option to save label data corresponding to the two-dimensional barcode and saving the label data in a selected one of a plurality of file formats. The plurality of reagent parameters includes at least one of a product name, a concentration, a formulation, a total volume, an expiration, a catalog number, a vendor, a lot number, a batch number, and a note. The method may include storing the identification in a datastore and associated with a template. The method may include selecting a label format from a plurality of label formats, where the act of printing the label includes printing the label to correspond with the label format. The method may include: selecting a scan barcode action; prompting the user with a label-scan request; scanning a barcode of a label; and selecting an option to save label data corresponding to the two-dimensional barcode. The method may include saving the label data in a selected one of a plurality of file formats.

The method may include storing the plurality of reagent parameters as a template in a datastore. The method may include providing a text string configured to be readable on the label adjacent to the two-dimensional barcode, where the act of printing the label includes printing the text string adjacent to the two-dimensional barcode. The method may include: selecting the create aliquot labels action; prompting the user with a label-scan request; scanning a preexisting label of a reagent in response to the label-scan request; identifying the sample based upon the identification embedded within the preexisting label; retrieving a plurality of reagent parameters corresponding to the sample; prompting the user with a request for at least one aliquot parameter including a number of aliquots; receiving the at least one aliquot parameter including the number of aliquots; embedding within the two-dimensional barcode the at least one aliquot parameter; adding a second text string configured to be readable on the label adjacent to the two-dimensional barcode; and printing a plurality of aliquot labels corresponding to the number of aliquots, where the plurality of labels includes the label. The method may include storing the plurality of reagent parameters, where the identification corresponds to the sample and the at least one aliquot parameter.

The at least on aliquot parameter can be a volume of fluid remaining, the method having notifying the user if the volume of fluid remaining is below a threshold for a predetermined number of aliquots. The method may include: storing the number of aliquots in a database; storing a number of aliquots remaining in the database; and notify the user if the number of aliquots remaining is below a predetermined threshold. The method may include directing the user to a product page link in response to the number of aliquots remaining below a predetermined threshold. The method may include automatically re-ordering the reagent in response to the number of aliquots remaining below a predetermined threshold. The method may include: selecting an option to save label data corresponding to the two-dimensional barcode; and saving the label data in a selected one of a plurality of file formats. The method may include: retrieving a plurality of reagent parameters corresponding to the preexisting label of the reagent; and embedding within the two-dimensional barcode the plurality of reagent parameters.

The method may include: selecting a modify two-dimensional barcode action; providing the plurality of reagent parameters; and modifying at least one of the plurality of reagent parameters, where the plurality of aliquot labels are printed in accordance with the modified at least one of the plurality of reagent parameters. The method may include: selecting an option to save label data corresponding to the two-dimensional barcode; and saving the label data in a selected one of a plurality of file formats. The method may include: selecting a scan barcode action; prompting the user with a label-scan request; scanning at least one of the plurality of aliquot labels; selecting a deduct action; prompting the user with a deduct amount and a number deducted of aliquots; and deducting the deduct amount from the number of aliquots. The method may include printing an updated plurality of aliquot labels corresponding to the number of deduced aliquots, where each of the updated plurality of aliquot labels embeds an updated aliquot volume in a respective two-dimensional barcode on a respective one of the updated plurality of aliquot labels. The method may include: selecting an option to save label data corresponding to the two-dimensional barcode; and saving the label data in a selected one of a plurality of file formats. The selected action is a sample history action. The method may include: providing a create new sample-history label action; and providing an update sample history label action.

The method may include: selecting the create new sample-history label action; providing a sample history label format; adding a first text string configured for embedding within the two-dimensional barcode; adding a second text string configured to be readable on the label adjacent to the two-dimensional barcode; and printing the label with the two-dimensional barcode on an adhesive material using a printer. The method may include adding a timestamp to one of the first text string and the second text string corresponding to a create time of the label. The method may include: selecting an option to save label data corresponding to the two-dimensional barcode; and saving the label data in a selected one of a plurality of file formats. The method may include: selecting the create new sample-history label action; providing a sample history label format; adding a text string associated with the identification; and storing the text string associated with the identification in a database. The method may include: selecting the update sample history label action; updating a text string associated with the identification; and storing the updated text string associated with the identification in a database.

The method may include: selecting the update sample history label action; prompting the user with a label-scan request; scanning a preexisting label in response to the label-scan request; identifying the sample based upon the identification embedded within the preexisting label; adding a second text string for appending to a first text string, where the first and second text string are configured for embedding within the two-dimensional barcode; and adding a third text string configured to be readable on the label adjacent to the two-dimensional barcode. The method may include adding a timestamp to one of the second text string and the third text string corresponding to an update time of the label. The method may include: selecting an option to save label data corresponding to the two-dimensional barcode; and saving the label data in a selected one of a plurality of file formats. The method may include scanning a label, where an action for selection of the plurality of actions for selection by the user is an action to duplicate the scanned label and the label is at least one duplicated label. The method may include: displaying at least one parameter corresponding to the scanned label; and prompting the user for modification of the at least one parameter, where the at least one duplicated label is printed in accordance with the modified at least one parameter. The method may include: scanning a first label of the sample; scanning a second label of another sample; selecting a mixture action; receiving a plurality of parameters corresponding to the mixture action; and embedding within the two-dimensional barcode the plurality of parameters corresponding to the sample. The method may include automatically generating at least one of the plurality of parameters corresponding to the mixture action.

The method may include saving a laboratory recipe corresponding to the plurality of parameters corresponding to the mixture action. The method may include determining an amount of a particular reagent should be added into a mixture based upon the mixture action to thereby achieve a predetermined concentration. The method may include embedding a plate map within the two-dimensional barcode. The method may include: scanning a label, where an action for selection of the plurality of actions for selection by the user is an action to dilute the sample corresponding to the scanned label; receiving a dilution amount; and embedding within the two-dimensional barcode the dilution amount corresponding to the sample. The method may include: scanning an equipment label; scanning a label corresponding to the sample; and storing a location of the sample as being stored within an equipment associated with the equipment label. The method may include storing the location on an RFID tag associated with at least one of the sample and the equipment. The method may include: scanning an equipment label; scanning a label corresponding to the sample; and storing an association of the sample with an equipment associated with the equipment label. The method may include: scanning a label corresponding to the sample; and displaying an indication of an expiration status corresponding to the sample. The method may include automatically supplying a link to the user corresponding to ordering a replacement of the sample when the indication corresponds to an expired sample. The method may include setting an expiration reminder corresponding to the expiration status. The method may include automatically setting an expiration reminder corresponding to the sample. The method may include: selecting a globally harmonized label creation action; searching for a reagent in a catalog; selecting the reagent; and retrieving a predetermined label format corresponding to the reagent. The two-dimensional barcode may correspond to the predetermined label format. The method may include: scanning a label; and retrieving a predetermined template corresponding to the label. The predetermined template may correspond to a globally harmonized label and the two-dimensional barcode may correspond to the predetermined template.

The sample in some embodiments may be a cell line, the method may include selecting a cell line action, where the two-dimensional barcode corresponds to the cell line action. The cell line action can be one of freezing, thawing, and storing the cell line. The method may include storing at least one of a cell density, a media, a supplement type, and a starting passage number corresponding to the cell line. The method may include receiving at least one of a cell count, a desired concentration, and a number of cryovials for embedding within the two-dimensional barcode.

The method may further include determining a volume of a cell line mixture to add to a cryovial; and determining an index for the cryovial. The method may include: receiving at least one reagent parameter; receiving a label format; generate a template corresponding to the label format the at least one reagent parameter; and saving the template for reuse. The identification can be a unique identification. The two-dimensional barcode encodes the sample workflow data in a standardized two-dimensional barcode format. The method may include selecting a label format from among a plurality of label formats, where each label format of the plurality of label formats is configured to attached to respective container type. The method may be implemented on a computer using an operative set of processor executable instructions configured to cause the computer to realize the method. Implementations of the described techniques may include hardware, a method or process, or a computer tangible medium.

In one general aspect, a device may include one or more processors configured to: provide a plurality of actions for selection by a user; select an action of the plurality of actions; determine an identification corresponding to a sample; embed within a two-dimensional barcode the identification and workflow data corresponding to the sample; and print a label with the two-dimensional barcode on an adhesive material using a printer. Other embodiments of this aspect include corresponding computer systems, apparatus, and computer programs recorded on one or more computer storage devices, each configured to perform the actions of the methods.

Implementations may include one or more of the following features. The device may save the workflow data as a string in human-readable format. The workflow data may include information about the experimental utilization of the sample. The one or more processors may be further configured to a guided menu configured to guide the user to input the workflow data. The selected action is an aliquot action. The one or more processors are further configured to: provide a create reagent label action; and provide a create aliquot labels action. The one or more processors are further configured to: select the create reagent label action; prompt the user with a request for a plurality of reagent parameters; receive the plurality of reagent parameters; and embed within the two-dimensional barcode the plurality of reagent parameters corresponding to the sample.

The one or more processors are further configured to store the plurality of reagent parameters and the identification corresponding to the sample in a database. The one or more processors are further configured to: select an option to save label data corresponding to the two-dimensional barcode; and save the label data in a selected one of a plurality of file formats. The plurality of reagent parameters includes at least one of a product name, a concentration, a formulation, a total volume, an expiration, a catalog number, a vendor, a lot number, a batch number, and a note. The one or more processors are further configured to store the identification in a datastore and associated with a template.

The one or more processors are further configured to select a label format from a plurality of label formats, where the act of printing the label includes printing the label to correspond with the label format. The one or more processors are further configured to: select a scan barcode action; prompt the user with a label-scan request; scan a barcode of a label; and select an option to save label data corresponding to the two-dimensional barcode. The one or more processors are further configured to save the label data in a selected one of a plurality of file formats. The one or more processors are further configured to store the plurality of reagent parameters as a template in a datastore. The one or more processors are further configured to provide a text string configured to be readable on the label adjacent to the two-dimensional barcode, where the act of printing the label includes printing the text string adjacent to the two-dimensional barcode. The one or more processors are further configured to: select the create aliquot labels action; prompt the user with a label-scan request; scan a preexisting label of a reagent in response to the label-scan request; identify the sample based upon the identification embedded within the preexisting label; retrieve a plurality of reagent parameters corresponding to the sample; prompt the user with a request for at least one aliquot parameter including a number of aliquots; receive the at least one aliquot parameter including the number of aliquots; embed within the two-dimensional barcode the at least one aliquot parameter; add a second text string configured to be readable on the label adjacent to the two-dimensional barcode; and print a plurality of aliquot labels corresponding to the number of aliquots, where the plurality of labels includes the label.

The one or more processors may be configured to store the plurality of reagent parameters, where the identification corresponds to the sample and the at least one aliquot parameter. The one or more processors, when the at least on aliquot parameter is a volume of fluid remaining, the method, are configured to notify the user if the volume of fluid remaining is below a threshold for a predetermined number of aliquots. The one or more processors may be configured to: store the number of aliquots in a database; store a number of aliquots remaining in the database; and notify the user if the number of aliquots remain is below a predetermined threshold. The one or more processors may be configured to direct the user to a product page link in response to the number of aliquots remaining below a predetermined threshold. The one or more processors may be configured to automatically re-order the reagent in response to the number of aliquots remaining below a predetermined threshold. The one or more processors may be configured to: select an option to save label data corresponding to the two-dimensional barcode; and save the label data in a selected one of a plurality of file formats. The one or more processors may be configured to: retrieve a plurality of reagent parameters corresponding to the preexisting label of the reagent; and embed within the two-dimensional barcode the plurality of reagent parameters.

The one or more processors may be configured to: select a modify two-dimensional barcode action; provide the plurality of reagent parameters; and modify at least one of the plurality of reagent parameters, where the plurality of aliquot labels are printed in accordance with the modified at least one of the plurality of reagent parameters. The one or more processors may be configured to: select an option to save label data corresponding to the two-dimensional barcode; and save the label data in a selected one of a plurality of file formats. The one or more processors may be configured to: select a scan barcode action; prompt the user with a label-scan request; scan at least one of the plurality of aliquot labels; select a deduct action; prompt the user with a deduct amount and a number deducted of aliquots; and deduct the deduct amount from the number of aliquots. The one or more processors may be configured to print an updated plurality of aliquot labels corresponding to the number of deduced aliquots, where each of the updated plurality of aliquot labels embeds an updated aliquot volume in a respective two-dimensional barcode on a respective one of the updated plurality of aliquot labels. The one or more processors may be configured to: select an option to save label data corresponding to the two-dimensional barcode; and save the label data in a selected one of a plurality of file formats. The selected action is a sample history action. The one or more processors may be configured to: provide a create new sample-history label action; and provide an update sample history label action. The one or more processors may be configured to: select the create new sample-history label action; provide a sample history label format; add a first text string configured for embedding within the two-dimensional barcode; add a second text string configured to be readable on the label adjacent to the two-dimensional barcode; and print the label with the two-dimensional barcode on an adhesive material using a printer. The one or more processors may be configured to add a timestamp to one of the first text string and the second text string corresponding to a create time of the label. The one or more processors may be configured to: select an option to save label data corresponding to the two-dimensional barcode; and save the label data in a selected one of a plurality of file formats.

The one or more processors may be configured to: select the create new sample-history label action; provide a sample history label format; add a text string associated with the identification; and store the text string associated with the identification in a database. The one or more processors may be configured to: select the update sample history label action; update a text string associated with the identification; and store the updated text string associated with the identification in a database. The one or more processors may be configured to: select the update sample history label action; prompt the user with a label-scan request; scan a preexisting label in response to the label-scan request; identify the sample based upon the identification embedded within the preexisting label; add a second text string for appending to a first text string, where the first and second text string are configured for embedding within the two-dimensional barcode; and add a third text string configured to be readable on the label adjacent to the two-dimensional barcode. The one or more processors may be configured to add a timestamp to one of the second text string and the third text string corresponding to an update time of the label. The one or more processors may be configured to: select an option to save label data corresponding to the two-dimensional barcode; and save the label data in a selected one of a plurality of file formats. The one or more processors may be configured to scan a label, where an action for selection of the plurality of actions for selection by the user is an action to duplicate the scanned label and the label is at least one duplicated label.

The one or more processors may be configured to: display at least one parameter corresponding to the scanned label; and prompt the user for modification of the at least one parameter, where the at least one duplicated label is printed in accordance with the modified at least one parameter. The one or more processors may be configured to: scan a first label of the sample; scan a second label of another sample; select a mixture action; receive a plurality of parameters corresponding to the mixture action; and embed within the two-dimensional barcode the plurality of parameters corresponding to the sample. The one or more processors may be configured to automatically generate at least one of the plurality of parameters corresponding to the mixture action. The one or more processors may be configured to save a laboratory recipe corresponding to the plurality of parameters corresponding to the mixture action. The one or more processors may be configured to determine an amount of a particular reagent should be added into a mixture based upon the mixture action to thereby achieve a predetermined concentration. The one or more processors may be configured to embed a plate map within the two-dimensional barcode.

The one or more processors may be configured to: scan a label, where an action for selection of the plurality of actions for selection by the user is an action to dilute the sample corresponding to the scanned label; receive a dilution amount; and embed within the two-dimensional barcode the dilution amount corresponding to the sample. The one or more processors may be configured to: scan an equipment label; scan a label corresponding to the sample; and store a location of the sample as being stored within an equipment associated with the equipment label. The one or more processors may be configured to store the location on an RFID tag associated with at least one of the sample and the equipment. The one or more processors may be configured to: scan an equipment label; scan a label corresponding to the sample; and store an association of the sample with an equipment associated with the equipment label. The one or more processors may be configured to: scan a label corresponding to the sample; and display an indication of an expiration status corresponding to the sample. The one or more processors may be configured to automatically supply a link to the user corresponding to ordering a replacement of the sample when the indication corresponds to an expired sample. The one or more processors may be configured to set an expiration reminder corresponding to the expiration status. The one or more processors may be configured to automatically set an expiration reminder corresponding to the sample. The one or more processors may be configured to: select a globally harmonized label creation action; search for a reagent in a catalog; select the reagent; retrieve a predetermined label format corresponding to the reagent; and the two-dimensional barcode corresponds to the predetermined label format. The one or more processors may be configured to: scan a label; and retrieve a predetermined template corresponding to the label where the predetermined template corresponds to a globally harmonized label and the two-dimensional barcode corresponds to the predetermined template.

The sample is a cell line, the method may include selecting a cell line action where the two-dimensional barcode corresponds to the cell line action. The cell line action is one of freezing, thawing, and storing the cell line. The one or more processors may be configured to store at least one of a cell density, a media, a supplement type, and a starting passage number corresponding to the cell line. The one or more processors may be configured to receive at least one of a cell count, a desired concentration, and a number of cryovials for embedding within the two-dimensional barcode. The one or more processors may be configured to: determine a volume of a cell line mixture to add to a cryovial; and determine an index for the cryovial. The one or more processors may be configured to: receive at least one reagent parameter; receive a label format; generate a template correspond to the label format the at least one reagent parameter; and save the template for reuse. The identification is a unique identification. The two-dimensional barcode encodes the sample workflow data in a standardized two-dimensional barcode format. The one or more processors may be configured to select a label format from among a plurality of label formats, where each label format of the plurality of label formats is configured to attached to respective container type. The method is implemented on a computer using an operative set of processor executable instructions configured to cause the computer to realize the method. The device is one of a computer, a smartphone, a PDA, a printer, a webserver, a server, a cloud server, a stand-alone printer, a handheld printer, a laptop, a wearable computer, and an embedded device. Implementations of the described techniques may include hardware, a method or process, or a computer tangible medium.

### BRIEF DESCRIPTION OF THE DRAWINGS

These and other aspects will become more apparent from the following detailed description of the various embodiments of the present disclosure with reference to the drawings wherein:

FIG. 1 shows a block diagram illustration of a cloud-based system for labeling and tracking laboratory samples in accordance with an embodiment of the present disclosure;

FIG. 2 show a block diagram illustration of a system for labeling and tracking laboratory samples in accordance with an embodiment of the present disclosure;

FIG. 3 shows a block diagram of a menu of actions on a graphical user interface in accordance with an embodiment of the present disclosure;

FIGS. 4A-4B show a flow chart diagram of a method to illustrate a create reagent label action in accordance with an embodiment of the present disclosure;

FIGS. 5A-5D show a flow chart diagram of a method to illustrate a create aliquot label action in accordance with an embodiment of the present disclosure;

FIG. 6 shows a flow chart diagram of a method to illustrate a create new sample-history label action in accordance with an embodiment of the present disclosure;

FIG. 7 shows a flow chart diagram of a method to illustrate an update sample-history label action in accordance with an embodiment of the present disclosure;

FIG. 8 shows a flow chart diagram of a method to illustrate a scan to export/save action in accordance with an embodiment of the present disclosure;

FIG. 9 shows a flow chart diagram of a method to illustrate a scan to deduct action in accordance with an embodiment of the present disclosure;

FIG. 10 shows a flow chart diagram of a method to illustrate a duplicate action in accordance with an embodiment of the present disclosure;

FIG. 11 shows a flow chart diagram of a method to illustrate an add action in accordance with an embodiment of the present disclosure;

FIG. 12 shows a flow chart diagram of a method to illustrate a dilution in accordance with an embodiment of the present disclosure;

FIG. 13 shows a flow chart diagram of a method to illustrate a scan-to-store action in accordance with an embodiment of the present disclosure;

FIG. 14 shows a flow chart diagram of a method to illustrate a scan-to-expiration action in accordance with an embodiment of the present disclosure;

FIG. 15 shows a flow chart diagram of a method to illustrate a globally harmonized label creation action in accordance with an embodiment of the present disclosure; and

FIGS. 16A-16B show a flow chart diagram of a method to illustrate cell line actions in accordance with an embodiment of the present disclosure.

### DETAILED DESCRIPTION

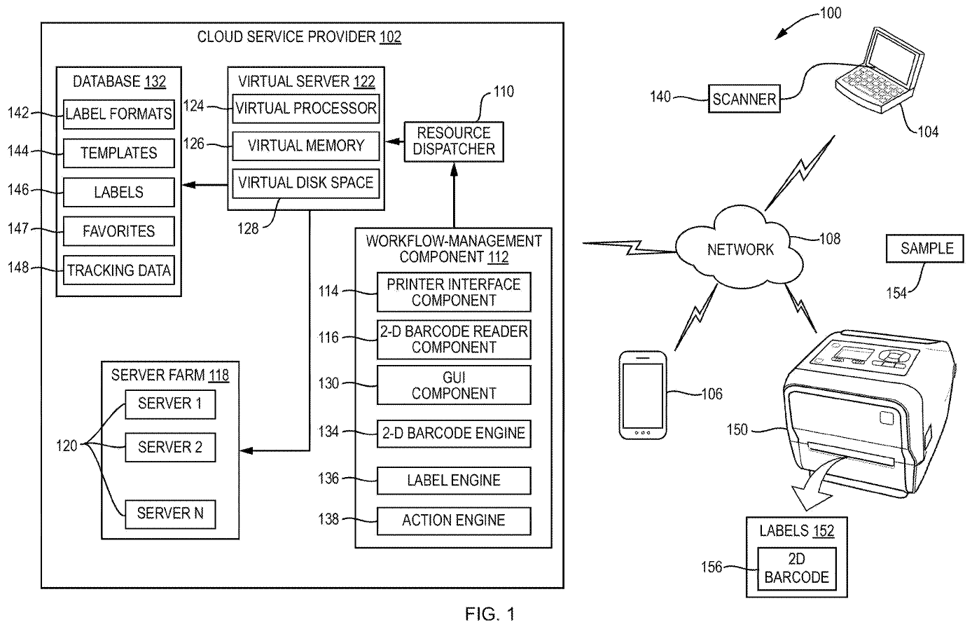

FIG. 1 shows a block-diagram illustration of a system 100 having a sever-client configuration for labeling and tracking laboratory samples in accordance with an embodiment of the present disclosure. The system 100 includes a cloud-service provider 102, a personal computer 104, a mobile device 106, and a printer 150. A workflow-management component 112 can provide label and tracking of laboratory samples 154 as described below in more detail.

The personal computer 104, the mobile device 106, and the printer 150 communicate with each other via a network 108. The network 108 may be Wi-Fi, ethernet, Bluetooth, etc. and may utilize the internet and associated protocols, such as TCP/IP. The network 108 may be a local area network, a wide-area network, a physical bus (such as a Universal Serial Bus), the internet, or some combination thereof.

The printer 150 may be an OEM printer for printing adhesives labels and may include a housing, label roll holder, a thermal printer head, a label cutter, and/or a power source (A/C power connector and/or battery). The printer 150 may dispense pre-perforated or scored label 152 rolls. The printer 150 may be controlled by a microcontroller, which receives data from a connected device, such as the smartphone 106 or the computer 104, which instructs the thermal printer head to print one or more labels 152. The printer 150 is suitable for printing labels 152 for labeling packages, products, biological samples, aliquot samples, reagent samples, or other items in various industries, such as healthcare, chemical or biological R&D laboratory settings. In some embodiments of the present disclosure, the mobile device 106 may be integrated with the printer 150 and/or the personal computer 104 may be integrated with the printer 150.

The personal computer 104 and mobile device 106 may interface with the cloud-service provider 102 to manage the labeling and tracking of the laboratory sample 15. In some embodiments, a specialize application for interfacing with the workflow-management component 112 may be used, such as a mobile application on the mobile device 106 or a desktop application on the personal computer 104. The communications may include transmitting data in HTML, XML, JSON, YAML, or any data format. The workflow-management component 112 may provide user-level accounts to individuals through a typical login mechanism. The workflow-management component 112 may be a web application, a webserver, a web service, etc. and may utilize one or more protocols to communicate data. The cloud service provider 102 may provide software updates to the printer 150, in some embodiments.

The system 100 includes a scanner 140. The scanner 140 may be mobile or fixed and may be wired or wireless. The scanner 140 is shown as being connected to the computer 104, but in some embodiments, it may be connected to the mobile device 106. In yet other embodiments, a camera of the computer 104 or the mobile device 106 may be the scanner 140.

The scanner 140 may utilize a laser, camera, or image sensor to read the two-dimensional barcode 156. The scanner 140 may include a processor that can decode the information stored on the two-dimensional barcode. The scanner 140 may also include a built-in display to show the scanned information to the user. The scanner 140 may include a trigger button or other mechanism for initiating the scanning process. In some embodiments, the scanner 140 may include the ability to scan multiple barcodes 156 in different rotational orientations, off-center orientations, or lighting conditions and be able to operate in less-than-ideal conditions (e.g., the scanner 140 may be in motion when scanning, etc.).

In some embodiments, the scanner 140 may be a RFID reader, an NFC reader, an optical scanner, or a combination of scanners. In yet additional embodiments, the scanner 140 may be two or more scanners of an RFID reader, an NFC reader, an optical scanner, or some combination thereof.

The description of the 2D barcode 156, including the claims, in other embodiments may be implemented by an RFID tag, either active or passive, or an NFC tag. The tag may be used in place of the 2D barcode 156, may be used in addition to the 2D barcode, may be integrated together with the 2D barcode 156 into a single adhesive, etc. The information stored on an RFID tag or NFC tag may be the same as the information stored on the 2D barcode 156; In yet further embodiments, the information stored within the RFID tag may include the same information as stored on the 2D barcode 156 along with other information, such as history information or more detailed information etc.

The cloud-service provider 102 provides a workflow-management component 112, which may be or include a webpage, a webapp, a program for download and execution on the computer 104 or the mobile device 106. The workflow-management component 112 facilitates the tracking, management, and the printing of labels 152 for samples for use in a laboratory setting. The kinds of workflow managed may include workflows focused toward academic and/or biopharma researchers in the cell culture, analytical, and molecular research areas, for example. For example, the workflow-management component 112 may instruct the printer 150 to print a two-dimensional barcode 156 on a label 152 that may be secured to the sample 154 for tracking. The two-dimensional barcode 156 may be scanned by the scanner 140 for identification and/or retrieval of data embedded within the two-dimensional barcode 156.

More particularly, the label 152 may include the two-dimensional barcode 156 configured to embed information therewithin to facilitating the tracking of the sample 154. The two-dimensional barcode 156 may store data corresponding to the sample 154 in plaintext using a widely recognized two-dimensional barcode format. In some specific embodiments, any device capable of reading the two-dimensional barcode 156 can determine any history, tracking, or other data parameters related to the sample 152 by reading the information in plaintext using any text viewer (e.g., an ASCII viewer). The two-dimensional barcode 156 may utilize the DataMatrix, QR Code, PDF 417, etc. format, which include both horizontal and vertical aspects for increased capability of data storage relative to 1D barcodes. Globally harmonized GS1 type two-dimensional barcode (for example, GS1 QR, GS1 DataMatrix) formats can be used to store the data in the two-dimensional barcode 156 in a standardized format for inventory management purposes and for streamlining product information.

Utilizing the two-dimensional barcode 152, a user can follow the laboratory sample 154 through their scientific workflows, retaining sample 154 information through procedures such as reagent aliquoting, sample history, creating mixtures and reactions for experimentation, tracking expiration dates, making dilutions, and storing reagents in specific locations among other procedures related to sample 154 management. The workflow-management component 112 can guide laboratory users specifically on how to best utilize the two-dimensional barcodes 156 for their specific applications and workflows. For example, the workflow-management component 112 can implement inventory management practices for bulk consumables and reagents. Thus, the workflow-management component 112 can be used to track and manage specific samples 154 within workflows (for example, a flask of growing cells, an aliquot of Penn/Strep, a buffer made in-house, a serial dilution of a drug, or a digestion reaction in a beaker of microplastics, etc.).

The workflow-management component 112 includes a printer interface component 114, a two-dimensional barcode reader component 116, a GUI component 130, a two-dimensional barcode engine 134, a label engine 136, and an action engine 138.

The printer interface component 114 may instruct the printer 150 to print one or more labels 152 configured for adhesively securing to one or more samples 154. The printer interface component 114 may include a print driver, communication capabilities (such as TCP/IP interface) and/or may be a button on a webpage that initiates a standardized print function on a web browser.

The two-dimensional barcode reader component 116 can interpret the data stored in the two-dimensional barcode 156. The two-dimensional barcode reader component 116 uses algorithms to decode the pattern of the two-dimensional barcode 156 into a data format readable by the computer 104, such as text, numbers, or binary data. Additionally or alternatively, the two-dimensional barcode reader component 116 may convert the data from the two-dimensional barcode into a variety of formats, including GS, PDF, DOC, DOCX, XLS, XLSX, JSON, CSV, XML, YAML, a markup language, etc.

The GUI component 130 can render a display for use by the computer 104 and/or the mobile device 106. The GUI component 130 may be a webpage-based provider, such as flask, an HTML server, a web framework, etc. The GUI component 130 may provide widgets, information, buttons, options, and menus to thereby facilitate a user's interaction with the workflow-management component 112.

The two-dimensional barcode engine 134 can generate a two-dimensional barcode 156 with a pattern that encodes data in a scanner 140 readable format. The two-dimensional barcode engine 134 can output the generated two-dimensional barcode 156 as an image file, a pdf file, or other format suitable for printing on one or more labels 152.

The label engine 136 can load, store, and save labels 146 in a database 132. For example, the label engine 1136 can store, load, or save label formats 142 that are the blank file formats that may represent a label 152 prior to printing on it. The label engine 136 can export the label formats 142 to one or more file formats. The label engine 136 can also, create, save, or retrieve templates 144, which is a label format 142 that has been filled in with data entered by a user. Additionally or alternatively, the label engine 136 can save favorites 142, including saving or indicating a label format 142 as a favorite 147 or a template 144 as a favorite 147. In some embodiments of the present disclosure, a favorite 147 may be applied to actions, menu selections, macros, etc. For example, a user can press button 332 at anytime a two-dimensional barcode is in the GUI to save the filled out label as a favorite template. Also, a user can export and/or save a label or template at any time by pressing the button 334 to scan a label which will appear on the screen such that a use can export or save the label and/or its data.

The workflow-management component 112 may also include an action engine 138. The action engine 138 may control the GUI component 130 and/or direct other components to execute instructions based upon a workflow initiated by a user. The action engine 138 may receive events, such as a mouse click, button press, or GUI widget interaction to initiate a routine, series of steps, or series of acts. For example, the action engine 138 may guide a user step-by-step on how to implement a particular workflow related to a sample 154 when performing a type of laboratory operation. The action engine 138 may execute an action from the menu of actions 300 of FIG. 3, further described below.

A resource dispatcher 110 may dispatch requests to perform an action to one or more virtual servers 122, each of which has a virtual processor 124, a virtual memory 126, and a virtual disk space 128. The virtual servers 122 can be executed on one or more servers 120 on a server farm 118 as dispatched and activated by the resource dispatcher 110.

The workflow-management component 112 can track the use and laboratory actions on the sample 154 by storing the history of the sample 154 within the database 132 as tracking data 148. The tracking data 148 may include chain of title information, chain of custody information, dilution history, temperature history, storage history, freeze history, aliquoting history, reagent history, reaction history, or other tracking information. In some embodiments, the workflow-management component 112 queries the database 132 to present data to a user via the GUI component 130. The workflow-management component 112 can also store technical or diagnostic information.

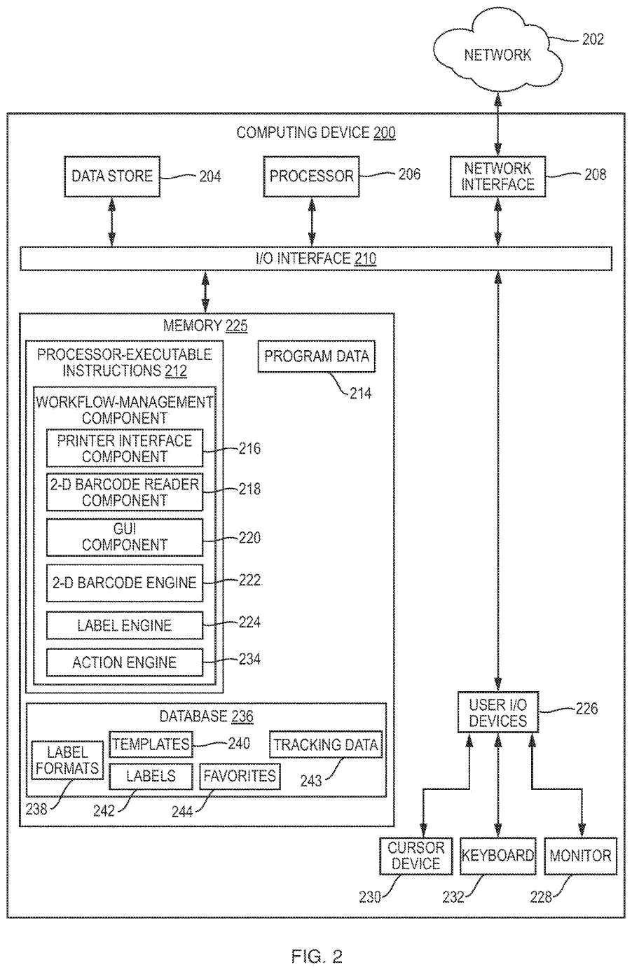

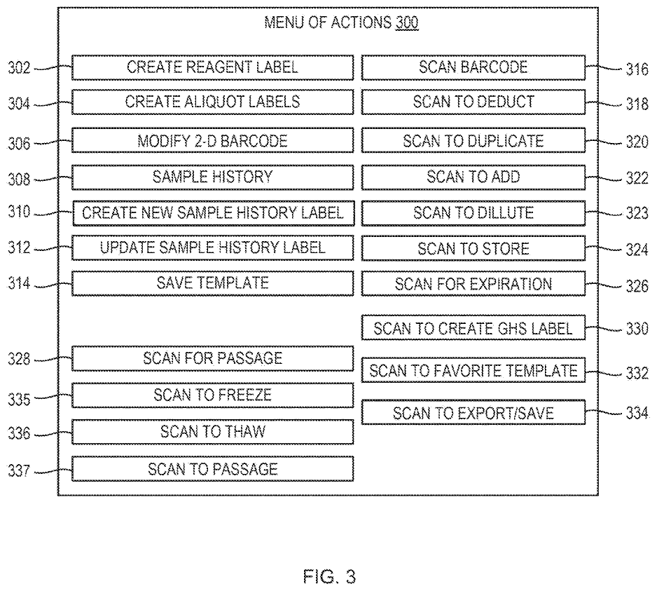

FIG. 2 shows a block-diagram illustration of a computing device 200 for labeling and tracking laboratory samples 154 in accordance with an embodiment of the present disclosure. The computing device 200 of FIG. 2 may be the computer 104 or mobile device 106 of FIG. 1. The computing device 200 includes an I/O interface 210 to communicate therewithin. The computing device 200 includes a data store 204, a processor 206, a network interface 208, a memory 225, and user I/O devices 226. The data store 204 stores data and may be a hard drive, flash drive, thumb drive, volatile memory, non-volatile memory, semi-volatile memory etc. The processor 206 can execute one or more processor-executable instructions 212, which may be stored in the data store 204 and/or the memory 225. For example, the processor 206 can execute processor-executable instructions 212 stored in memory 225 that was retrieved from the data store 204. The memory 225 also includes program data 214 that may include information related to the processor-executable instructions 212. The computing device 200 may include user I/O devices 226, such as a cursor device 230 (e.g., touchscreen or mouse), a keyboard 232 (virtual or physical), and/or a monitor 228 (which may be a touchscreen). The computing device 200 communicates with the network 202 via a network interface 208.

Although the computing device 200 of FIG. 2 may be used as part of the system 100 of FIG. 1, in some embodiments, the label and tracking functionality may reside wholly within the computing device 200 of FIG. 2. For example, the workflow-management component 112 of FIG. 1 may reside within the processor-executable instructions 212 of FIG. 2 as workflow-management component 215. For example, the printer interface component 216, the two-dimensional barcode reader component 218, the GUI component 220, the two-dimensional barcode engine 222, the label engine 223, and the action 234 of FIG. 2 may have the same or similar functionality as the printer interface component 114, the two-dimensional barcode reader component 116, the GUI component 130, the two-dimensional barcode engine 134, the label engine 136, and the action engine 139 of FIG. 1, respectively. The database 236 may be similar to the database 132 of FIG. 1. The database 236 may, for example, be an SQLite 3 database embedded on the computing device 200.

Thus, in some embodiments the workflow-management component 112, 215 may reside wholly on a local device (such as computer 104, device 106, printer 150, etc.) may be partially within a cloud service provider 102, and/or may be organized in a hybrid local and cloud configuration. In some embodiments, the workflow-management component 215 may be an application, may be executed on the computer 104, the device 106, the printer 150, the cloud service provider 102, the computing device 200, etc. or some combination thereof.

The database 236 of FIG. 2 may be like or identical to the database 132 of FIG. 1. That is, the label formats 238, the templates 240, the labels 242, the favorites 244, and the track data 243 of FIG. 2 may be similar or the same as the label formats 142, the templates 144, the labels 146, the favorites 147, and the tracking data 148 of FIG. 1, respectively.

FIG. 3 shows a block diagram of a menu of actions 300 on a graphical user interface in accordance with an embodiment of the present disclosure. The menu of actions 300 includes buttons 302-334. The buttons 302-334 may be menu items, may be clickable buttons, may be drop-down buttons, may be a menu hierarchy, may be conditional, etc. For example, some of the buttons 302-344 may be preconditioned on selecting previous buttons and/or taking previous actions (such as scanning a barcode without being prompted, etc.).

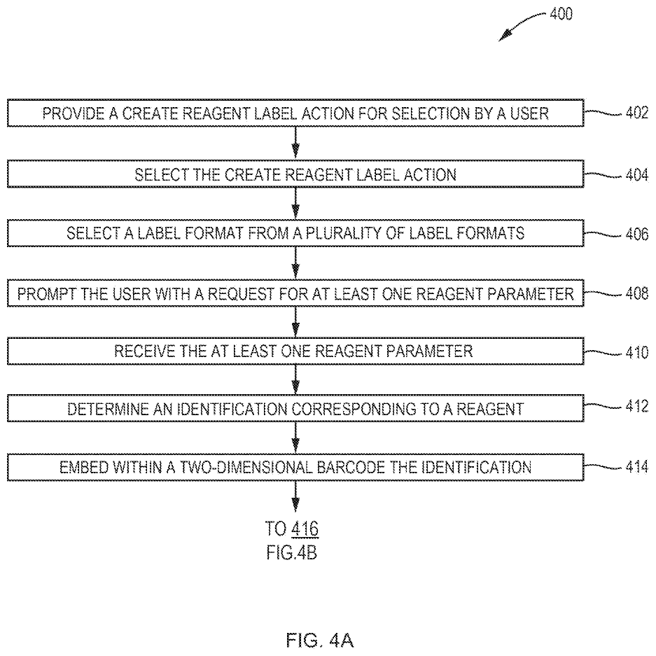

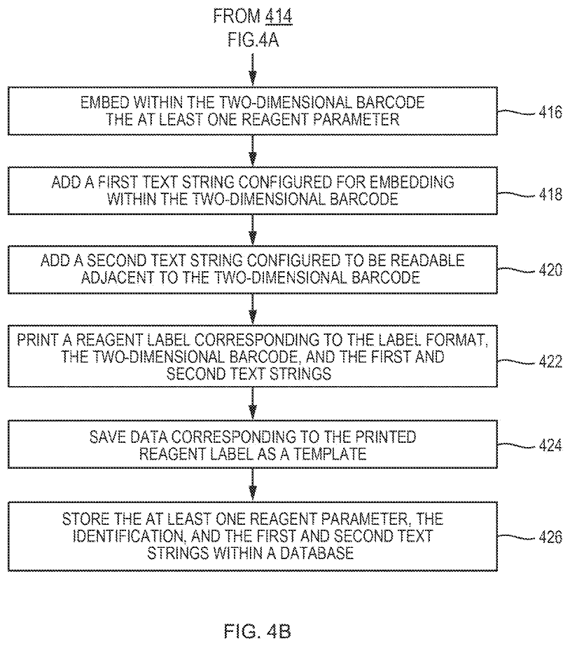

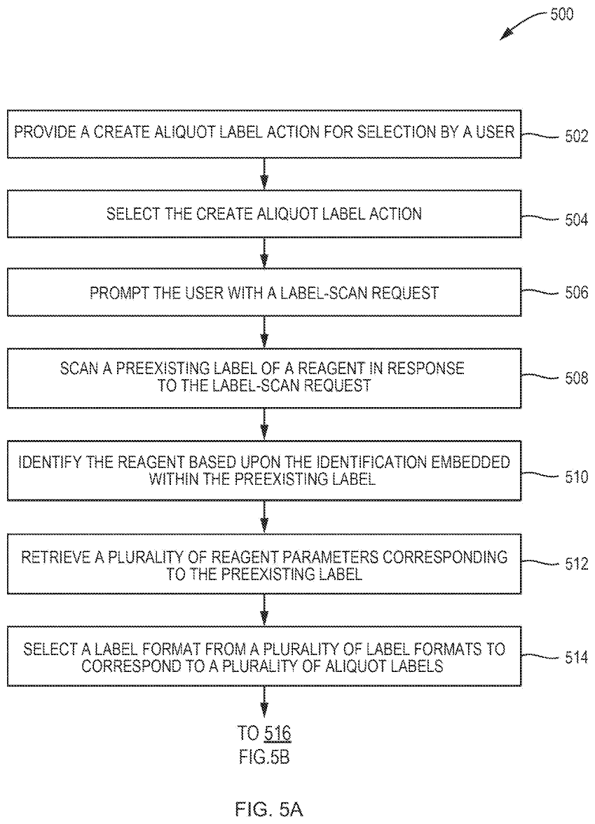

Button 302 initiates a create reagent label action. According to an example, one or more process blocks of FIG. 4A-4B may be performed by the computing device 200 in response to the create reagent label action button 302 being pressed. The process 400 may include providing a create reagent label action for selection by a user (block 402). Thus, the button 302 of FIG. 3 may be presented to a user for selection in Act 402. In some embodiments, a user can select a predetermined reagent item, e.g., using a SKU#, to automatically populate information to create new labels 152.

The computing device 200 may provide a create reagent label action for selection by a user by presenting the button 302 for selection within a menu hierarchy. The process 400 shown in FIG. 4A may include selecting the create reagent label action (block 404). For example, a user may click on the button 302 using the computing device 200 which then causes the computing device 200 to initiate the process 400. The action engine 234 may initiate, control, and/or orchestrate the process 400.

The process 400 may include selecting a label format from a plurality of label formats 238 (block 406). The process 400 prompts the user with a request for at least one reagent parameter (block 408). A popup box may pop up on the GUI to allow a user to enter in one or more reagent parameters, for example. The process 400 may receive the at least one reagent parameter (block 410), e.g., from the pop-up dialog. The process 400 determines an identification corresponding to a reagent (block 412). The identification may be from a database 236, may have been embedded within a two-dimensional barcode from a scanned barcode or may be manually entered. The process 400 may include embedding within a two-dimensional barcode the identification (block 414). This may include generating a two-dimensional bar code by the two-dimensional barcode engine 222 which is then used by the label engine 224 to generate an image of the label 152 suitable for printing by the printer 150.

The computing device 200 may embed within a two-dimensional barcode 156 the identification, as described above. The process 400 may include embedding within the two-dimensional barcode 156 the at least one reagent parameter (block 416). The process 400 may include adding a first text string configured for embedding within the two-dimensional barcode (block 418). For example, any additional text may be added to two-dimensional barcode 156 that may be used by a user that reads the two-dimensional barcode 156. Act 420 adds a second text string configured to be readable adjacent to the two-dimensional barcode (block 420). For example, there may be a section on the label 152 to place raw text on the label 152. Act 422 prints a reagent label corresponding to the label format, the two-dimensional barcode 156, and the first and second text strings (block 422). The process 400 may include Act 242 of saving data corresponding to the printed reagent label as a template 240 (block 424). The computing device 200 may save the template 240 for later retrieval by a user. Act 426 stores at least one reagent parameter, the identification, and the first and second text strings within a database 236. The computing device 200 may store the at least one reagent parameter, the identification, and the first and second text strings within in the internal database 236, or in other embodiments, within an external database 132 (see FIG. 1)

It should be noted that while FIGS. 4A-4B shows example blocks of process 400, in some implementations, the process 400 may include additional blocks, fewer blocks, different blocks, or differently arranged blocks than those depicted in FIG. 4. Additionally, or alternatively, two or more of the blocks of process 400 may be performed in parallel.

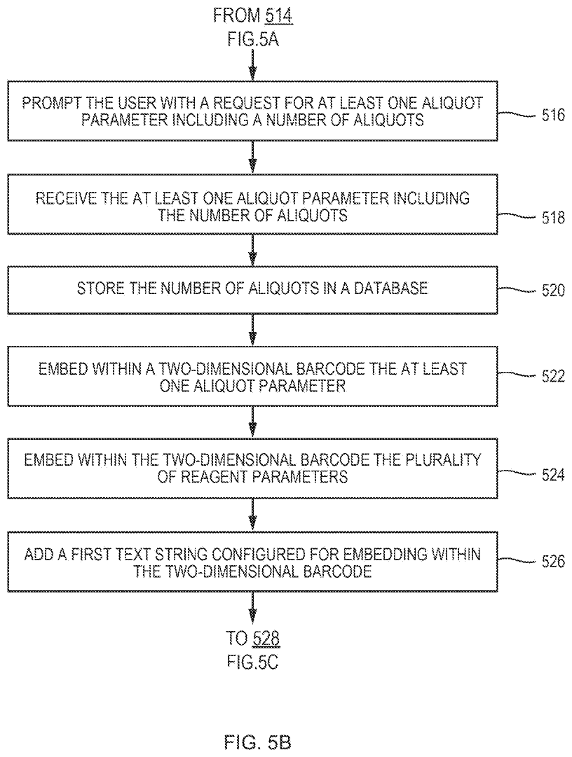

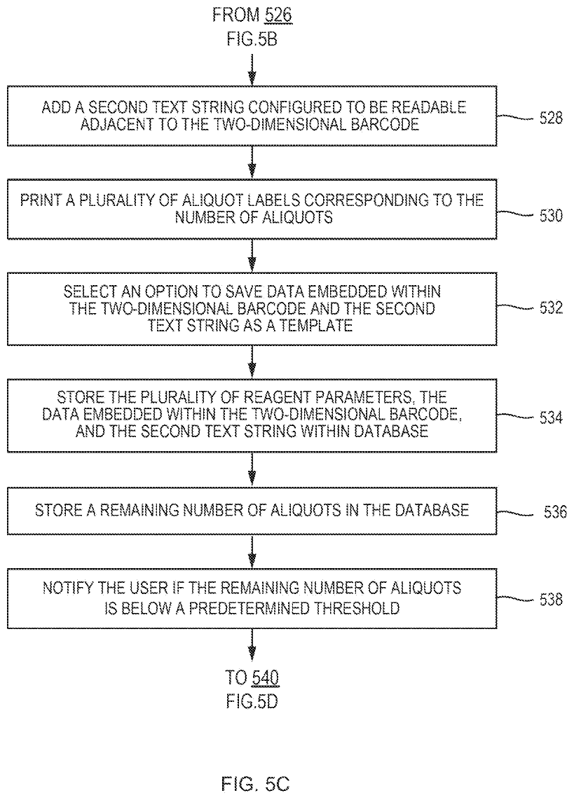

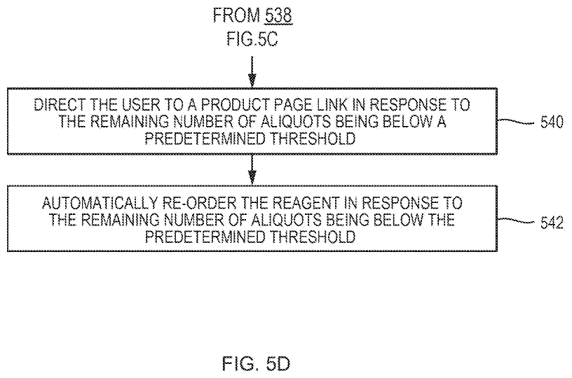

Referring again to FIG. 3, a user may select a create labels button 304 to initiate the aliquoting process. This may activate the action engine 234 to initiate the process 500 shown in FIGS. 5A-5D. The process 500 may be performed by the computing device 200, e.g., by the action engine 234 of the computing device 200.

The aliquoting workflow may include labeling incoming reagents, labeling aliquots, and/or the process of “aliquoting”, i.e., taking a portion of a relatively large volume reagent and placing a relatively small volume of it into a smaller vessel (for example, transferring ten, 1 mL aliquots from 20 ml bottle into ten, 2 mL tubes tubes). Aliquoting may be used to create a single-use reagent or/or to avoiding freeze-thaw cycles.

For example, smaller “aliquot” labels from one larger reagent label may be created from the reagent label that the user previously created. These labels may include a two-dimensional barcode 156 which contains information of the reagent which may be automatically transferred into a new smaller aliquot label, for example. Users can choose which label format 238 they would like for their reagent and aliquot labels.

Information may be entered by a user related to the aliquots, such as how many aliquots are expected to be made from the reagent. Multiple aliquot labels may be printed each corresponding to an aliquot label 152 configured to facilitate the tracking of all the aliquots including how many aliquots are left in inventory. The computing device 200, upon the amount getting “low”, may inform the user that new aliquots should be made and/or more reagents should be ordered.

Referring now to FIGS. 5A-5D, the process 500 may include act 503 that provides a create aliquot label action for selection by a user. This may include providing the button 304 for selection by a user. In Act 504, a user may select the create aliquot label action. Act 506 prompts the user with a label-scan request. Act 508 scans a preexisting label of a reagent in response to the label-scan request. For example, a user may use the scanner 140 attached to the computing device 200 to scan a preexisting label of a reagent in response to the label-scan request. Act 510 identifies the reagent based upon the identification embedded within the preexisting label. For example, the computing device 200 may retrieve the identify the reagent based upon the identification embedded within the two-dimensional barcode 156 of a reagent selected to aliquot.

Act 512 retrieves a plurality of reagent parameters corresponding to the preexisting label. The reagent parameters may be parameters embedded within the two-dimensional barcode 156 attached to the reagent and/or may be retrieved from the database 236. Act 514 selects a label format from a plurality of label formats 238 to correspond to a plurality of aliquot labels. That is, the computing device 200 may select a label format 238 from many label formats that are configured to facilitate tracking and management of aliquot samples 154.

Act 516 prompts the user with a request for at least one aliquot parameter including a number of aliquots. Act 518 receives the at least one aliquot parameter including a number of aliquots, which may have been entered by a user. If, for example, a reagent was going to be converted into 10 aliquots, the user would enter in the number 10 into the GUI of the computing device 200. Act 520 stores the number of aliquots in a database 235. Act 522 embeds within a two-dimensional barcode 156 the at least one aliquot parameter. The embedding within the two-dimensional barcode 156 may be within the memory 225. The number of aliquots may be embedded within the two-dimensional barcode as plaintext. Additionally or alternatively, an aliquot number may be embedded within the two-dimensional barcode. That is, the two-dimensional barcode engine 222 may build the two-dimensional barcode 156 within the memory 225 prior to printing. In Act 542, the process may embed within the two-dimensional barcode 156 the plurality of reagent parameters as well. In Act 526, a user may enter in a first text string that is added to the two-dimensional barcode 156. This may be plaintext that is added to the two-dimensional barcode within the memory 225, which is the label 152 attached to the sample 154.

As further shown in FIG. 5C, the process 500 may include adding a second text string configured to be readable adjacent to the two-dimensional barcode (block 528). The second text string may be text that is printed on the label 152 and may be readily readable. The process 500 may include printing a plurality of aliquot labels corresponding to the number of aliquots (block 530). In Act 532, a user may select an option to save data embedded within the two-dimensional barcode and the second text string as a template 240. For example, the computing device 200 may save data embedded within the two-dimensional barcode and the second text string as a template.

The process 500 may store the plurality of reagent parameters, the data embedded within the two-dimensional barcode 156, and the second text string within the database 236 (block 534). The remaining number of aliquots may be stored within the database 236 for later retrieval by the computing device 200 in Act 536. The computing device 200 may notify the user if the remaining number of aliquots is below a predetermined threshold in Act 538. For example, a pop-up alert may alert a user when the number of aliquots is low as predefined by a user within settings. The computing device 200 may direct the user to a product page link in response to the remaining number of aliquots being below a predetermined threshold (block 540). The computing device may automatically re-ordering the reagent in response to the remaining number of aliquots being below the predetermined threshold in Act 542.

It should be noted that while FIGS. 5A-5D shows example blocks of process 500, in some implementations, the process 500 may include additional blocks, fewer blocks, different blocks, or differently arranged blocks than those depicted in FIGS. 5A-5D. Additionally, or alternatively, two or more of the blocks of process 500 may be performed in parallel.

Referring again to FIG. 3, a user may select button 306 which initiates an action to modify a two-dimensional barcode 156 presented to the user prior to printing, during the workflow, or at any other time. The ability to modify a two-dimensional barcode manually may be available at all times, in some specific embodiments. A user may select button 308 which prompts a user to scan a two-dimensional barcode. After scanning, the entire sample history may be shown to the user. The sample history may be embedded within the two-dimensional barcode and/or may be retrieved from storage on the database. For example, if a sample was heated for 2 hours, this information might be documented within the two-dimensional barcodes and marked by time/date stamps. These data can be copied and pasted from the program, saved in a template format or exported into a PDF for image for recordkeeping purposes. Additionally or alternatively, sample history snapshots can be uploaded to and updated in a location in the cloud, where an practically infinite amount of data about the sample can be stored and accessed at any time and place. Further, the data may be linked to other equipment or experiments utilizing the cloud storage.

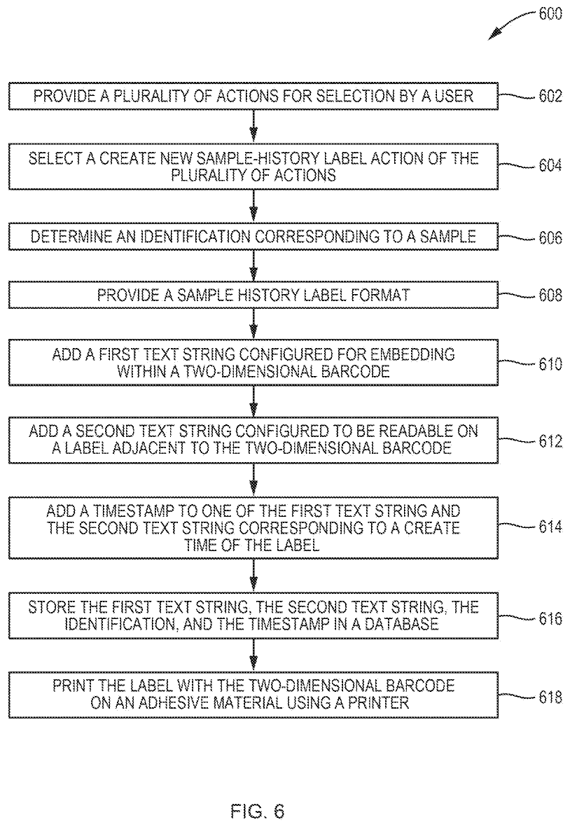

A user may also select the create sample history action by pressing button 310. By pressing button 310, a user initiates the process 600 shown in FIG. 6. The process 600 may be initiated by the action engine 138 of FIG. 1 or the action engine 234 of FIG. 2. FIG. 6 shows a flow chart diagram of a method 600 to illustrate a create new sample-history label action in accordance with an embodiment of the present disclosure. According to an example, one or more process blocks of FIG. 6 may be performed by the computing device 200.

Act 602 provides a plurality of actions for selection by a user. Act 604 selects a create new sample-history label action of the plurality of actions. This may be to generate a label for tracking within a laboratory environment. Act 606 determines an identification corresponding to a sample. Act 608 provides a sample history label format. In some embodiments, the two-dimensional barcode 156 may include the entire history of the sample 154. Act 610 adds a first text string configured for embedding within a two-dimensional barcode. This may be any arbitrary text entered in by the user. Act 612 adds a second text string configured to be readable on a label adjacent to the two-dimensional barcode. Act 614 adds a timestamp to one of the first text string and the second text string corresponding to a create time of the label. The timestamp may be ASCII added which includes a date/time text. As additionally shown in FIG. 6, the process 600 may include storing the first text string, the second text string, the identification, and the timestamp in a database (block 616). As also shown in FIG. 6, the process 600 may include printing the label with the two-dimensional barcode on an adhesive material using the printer 150 (block 618).

It should be noted that while FIG. 6 shows example blocks of process 600, in some implementations, the process 600 may include additional blocks, fewer blocks, different blocks, or differently arranged blocks than those depicted in FIG. 6. Additionally, or alternatively, two or more of the blocks of process 600 may be performed in parallel.

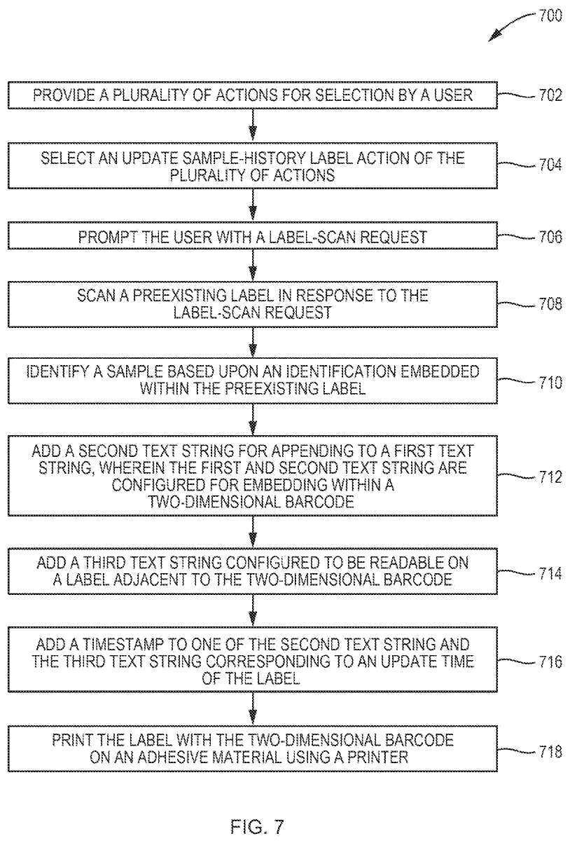

FIG. 7 shows a flow chart diagram of a method to illustrate an update sample-history label action in accordance with an embodiment of the present disclosure. According to an example, one or more process blocks of FIG. 7 may be performed by computer device 200.

As shown in FIG. 7, the process 700 may include providing a plurality of actions for selection by a user (block 702). As in addition shown in FIG. 7, the process 700 may include selecting an update sample-history label action of the plurality of actions (block 704). As also shown in FIG. 7, the process 700 may include prompting the user with a label-scan request (block 706). As further shown in FIG. 7, the process 700 may include scanning a preexisting label in response to the label-scan request (block 708). The process 700 may include identifying a sample based upon an identification embedded within the preexisting label (block 710). The process 700 may include adding a second text string for appending to a first text string, where the first and second text string are configured for embedding within a two-dimensional barcode (block 712). A third text string configured to be readable on a label adjacent to the two-dimensional barcode may also be added (block 714). The process 700 may include adding a timestamp to one of the second text string and the third text string corresponding to an update time of the label (block 716). Act 718 prints the label with the two-dimensional barcode on an adhesive material using a printer.

It should be noted that while FIG. 7 shows example blocks of process 700, in some implementations, the process 700 may include additional blocks, fewer blocks, different blocks, or differently arranged blocks than those depicted in FIG. 7. Additionally, or alternatively, two or more of the blocks of process 700 may be performed in parallel.

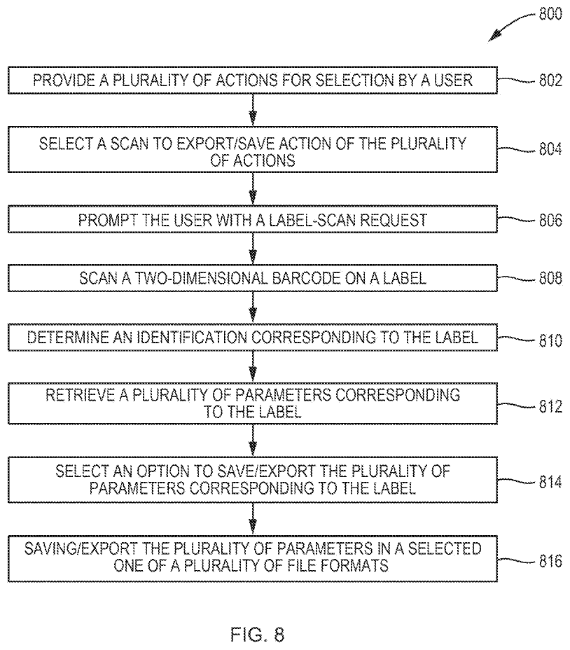

Referring again to FIG. 3, a user can select the button 324 to save a template. The button 324 can initiate the process 800 of FIG. 8. The action may be similar to entering in data a lab notebook or saving data to a location in file navigator for later viewing and/or analysis. The data may be embedded within the two-dimensional barcode, including the sample history, or another feature/functionality described herein, which may be exported to a file or stored in the cloud, in a PDF, MS word doc, txt file, or image format, etc. In some embodiments, this data is store automatically and does not need to be exported.

Furthermore, after selecting button 324, the method 800 may be initiated. FIG. 8 shows a flow chart diagram of the method 800 to illustrate a scan to export/save action in accordance with an embodiment of the present disclosure. According to an example, one or more process blocks of FIG. 8 may be performed by the computing device 200.

As shown in FIG. 8, the process 800 may include providing a plurality of actions for selection by a user (block 802). Act 804 selects a scan to export/save action of the plurality of actions. The process 800 may include prompting the user with a label-scan request (block 806). As further shown in FIG. 8, the process 800 may include scanning a two-dimensional barcode on a label (block 808). Act 810 determines an identification corresponding to the label. The process 800 may include retrieving a plurality of parameters corresponding to the label (block 812). As further shown in FIG. 8, the process 800 may include selecting an option to save/export the plurality of parameters corresponding to the label (block 814). The process 800 may include saving/exporting the plurality of parameters in a selected one of a plurality of file formats (block 816).

It should be noted that while FIG. 8 shows example blocks of process 800, in some implementations, the process 800 may include additional blocks, fewer blocks, different blocks, or differently arranged blocks than those depicted in FIG. 8. Additionally, or alternatively, two or more of the blocks of process 800 may be performed in parallel.

Referring again to FIG. 3, a user may select a scan barcode action 316. The scan barcode action 316 may simply allow certain buttons to be pressed after selection of the button 316. For example, after pressing button 316, all buttons that are logically selectable after scanning a barcode may become selectable, in some embodiments.

Another possible action in the menu of actions 300, is a button 318 to deduct, e.g., deduct from a reagent, an aliquot, and/or other samples. Workflow: inventory management of reagents, aliquots and other samples. The computing device 200 can keep track of aliquots and is able to notify and remind the user that the existing inventory of aliquots is getting “low” enough to either reorder a new reagent or make new aliquots, before running out. The user is able to set the number of aliquots that is considered “low” enough for them to make more aliquots, and the reminder can come in-app or through text messages and/or email. In some embodiments, the computing device 200 can help in replenishment of reagents that are getting “low” by directing the user to product page links, or automatically re-order reagents

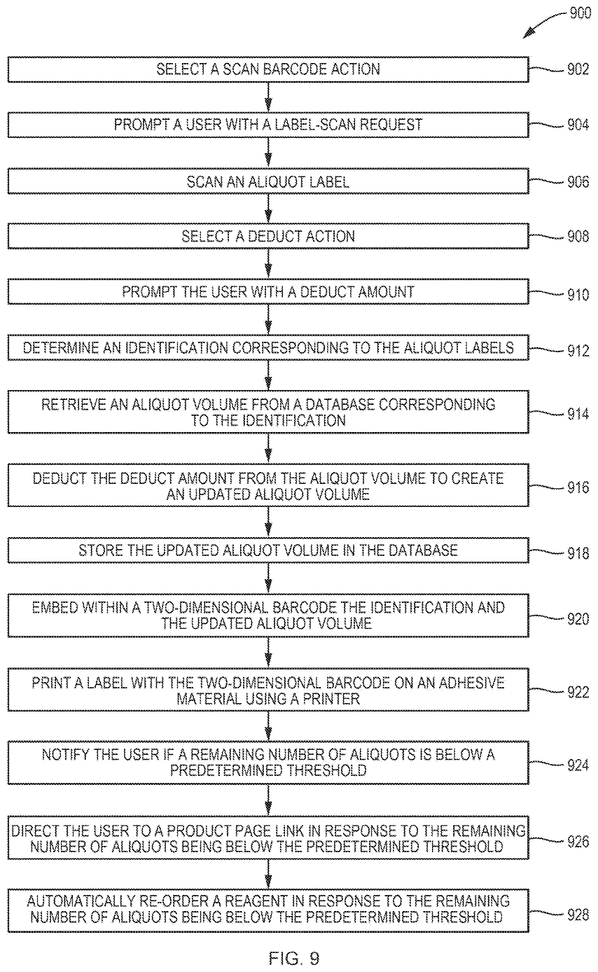

Referring again to FIGS. 3 and 9: The button 318 can cause the computing device 200 to initiate the method 900 of FIG. 9. FIG. 9 shows a flow chart diagram of a method 900 to illustrate a deduct action in accordance with an embodiment of the present disclosure. Thus, according to an example, one or more process blocks of FIG. 9 may be performed by the computing device 200 (see FIG. 2).

As shown in FIG. 9, the process 900 may include selecting a scan barcode action (block 902). Act 904 prompts a user with a label-scan request. In Act 906, a user scans an aliquot label and in Act 908 the user selects a deduct action. The deduct action may initiated by pressing the button 318. In Act 910, the method 900 prompts the user with a deduct amount. Here a user can enter in how much to deduct from a group of aliquots. In Act 912, the computing device 200 determines an identification corresponding to the aliquot labels and in Act 914 it retrieves an aliquot volume from a database that corresponding to the identification. Act 916 deducts the deduct amount from the aliquot volume to create an updated aliquot volume (e.g., for updating within the database) and Act 918 stores the updated aliquot volume in the database. Act 920 embeds within a two-dimensional barcode the identification and the updated aliquot volume. The process 900 prints a label with the two-dimensional barcode on an adhesive material using a printer (block 922). In Act 924, the user is notified if a remaining number of aliquots is below a predetermined threshold (block 924). Act 926 directs the user to a product page link in response to the remaining number of aliquots being below the predetermined threshold. Act 928 automatically re-orders a reagent in response to the remaining number of aliquots being below the predetermined threshold. Acts 926-928 are optional and can be configured within the settings of the software.

It should be noted that while FIG. 9 shows example blocks of process 900, in some implementations, the process 900 may include additional blocks, fewer blocks, different blocks, or differently arranged blocks than those depicted in FIG. 9. Additionally, or alternatively, two or more of the blocks of process 900 may be performed in parallel.

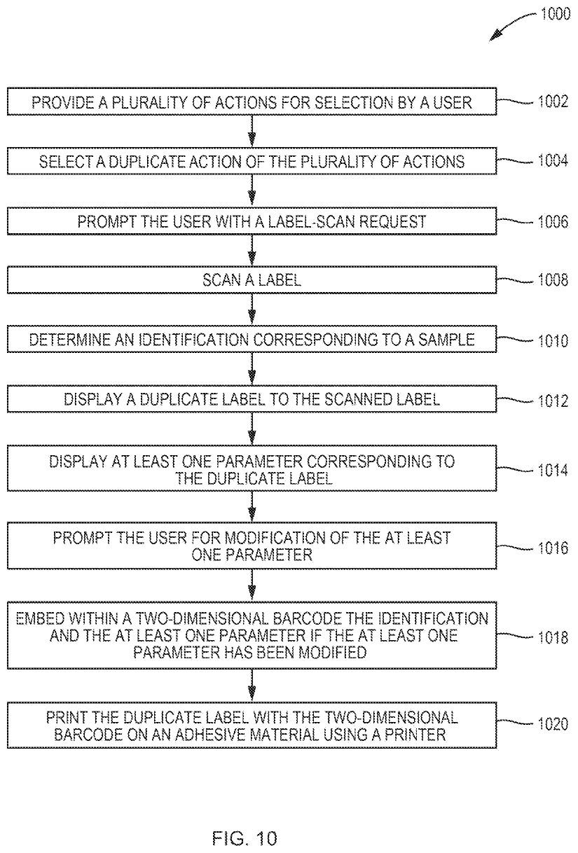

If a user wants to duplicate a label, the user may press button 320 to initiate the process within the computing device 200. That initiates the method 1000 shown in FIG. 10. That is, FIG. 10 shows a flow chart diagram of a method/process 10000 to illustrate a duplicate action in accordance with an embodiment of the present disclosure. According to an example, one or more process blocks of FIG. 10 may be performed by the computing device 200. Method 1000 may be used to create multiple labels that are exact copies and/or are modified before being printed. The user can be prompted to scan the existing barcoded label and the computing device 200 can exact a copy of the scanned label, with all of the sample information included within the barcode, which can then appear on the screen. The user can then print copies, make modifications, or copy/paste and export the label or the label's data for recordkeeping purposes.

Act 1002 provides a plurality of actions for selection by a user, e.g., by providing the menu of action 300 shown in FIG. 3. In Act 1004, a user selects a duplicate action of the plurality of actions. Act 1006 prompts the user with a label-scan request. The user scans a label (block 1008). The computing device in Act 1010 can determine an identification corresponding to a sample. Act 1012 displays a duplicate label to the scanned label and Act 1014 displays at least one parameter corresponding to the duplicate label. The user may be prompted for modification of the at least one parameter (block 1016). At Act 1018, the process 1000 embeds within a two-dimensional barcode the identification and the at least one parameter if the at least one parameter has been modified (block 1018). Act 1020 prints the duplicate label with the two-dimensional barcode on an adhesive material using a printer.

It should be noted that while FIG. 10 shows example blocks of process 1000, in some implementations, the process 1000 may include additional blocks, fewer blocks, different blocks, or differently arranged blocks than those depicted in FIG. 10. Additionally, or alternatively, two or more of the blocks of process 1000 may be performed in parallel.

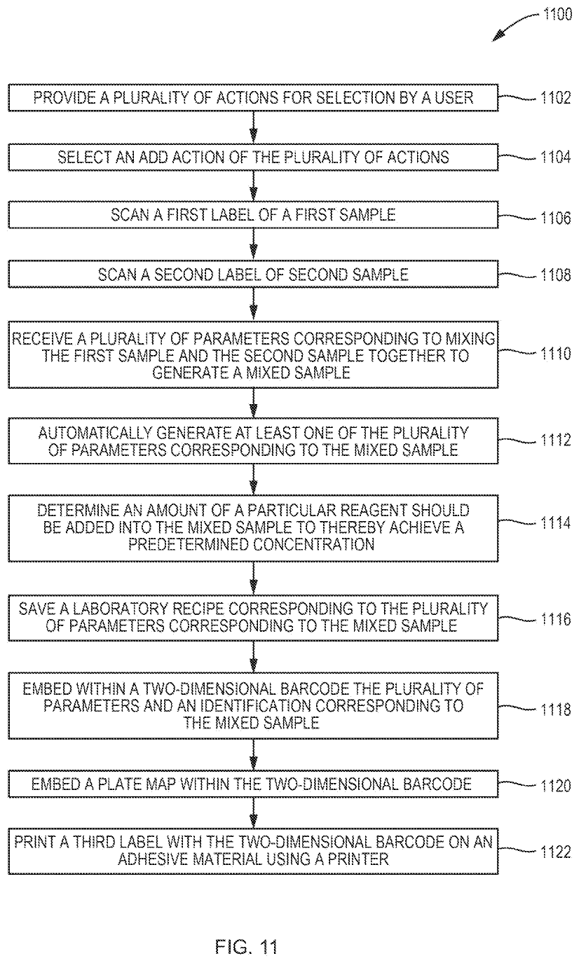

Referring to FIG. 3 and FIG. 11: A user may press the scan to add action 322 to initiate the process or adding to a sample, such as the method 1100 which illustrates an add action in accordance with an embodiment of the present disclosure. According to an example, one or more process blocks of FIG. 11 may be performed by the computing device 200.

For example, a user can create a new “mixture” in the laboratory, which could be a chemical reaction, a buffer or mobile phase reagent mixture, an experiment or otherwise, where multiple components (such as reagents, chemicals, buffers, aliquots, etc.) are mixed together and manipulated by scientific means. The computing device 200 may allow the user to scan multiple barcodes on each of the components being mixed together (or, if they do not have barcodes on one or more of the components, the user can manually enter the information for a particular component) and then combine this information together into a single new barcode for the overall mixture, reaction, etc. The computing device also can automatically populate the label text to provide identifiers of the experiment or mixture, which the user may edit if desired. The label can be printed and applied onto the vessel in which the mixture is being made. This label can be saved as a common “recipe” as a template for the next time this reaction/mixture is performed in the lab. Upon scanning a mixture label, the user may see information about each of the components and may save the information, copy/paste or export for recordkeeping purposes.

In yet additional embodiments, the computing device 200 calculates the final concentration or composition of each component in the mixture (for example, a mixture of 10% (v/v) FBS, 0.05 M sodium pyruvate, and 1 mM EDTA). The computing device may provide a calculator for determining how much of a particular reagent should be added into the mixture to produce the final concentration. In yet additional embodiments, the computing device also can include a plate map to be stored within the two-dimensional barcode that can be populated, saved, and exported by the user for recordkeeping purposes using the computing device.

As shown in FIG. 11, the method 1100 may include providing a plurality of actions for selection by a user (block 1102). In Act 1104, a user may select an add action of the plurality of actions. A user in Act 1106 can scan a first label of a first sample and scan a second label of second sample in Act 1108. The computing device 200 may receive a plurality of parameters corresponding to mixing the first sample and the second sample together to generate a mixed sample (block 1110). Receiving as used herein may be indicate that the information or data was received via user input, via database response, as found in a scanned barcode, and/or as received from a data file.

As also shown in FIG. 11, the process 1100 may include automatically generating at least one of the plurality of parameters corresponding to the mixed sample (block 1112). Act 1114 determines an amount of a particular reagent should be added into the mixed sample to thereby achieve a predetermined concentration. The process 1100 may also include saving a laboratory recipe corresponding to the plurality of parameters corresponding to the mixed sample (block 1116). Act 1118 may embed within a two-dimensional barcode the plurality of parameters and an identification corresponding to the mixed sample and Act 1120 may embed a plate map within the two-dimensional barcode. In Act 1122, the computing device prints a third label with the two-dimensional barcode on an adhesive material using a printer (block 1122).

It should be noted that while FIG. 11 shows example blocks of process 1100, in some implementations, the process 1100 may include additional blocks, fewer blocks, different blocks, or differently arranged blocks than those depicted in FIG. 11. Additionally, or alternatively, two or more of the blocks of process 1100 may be performed in parallel

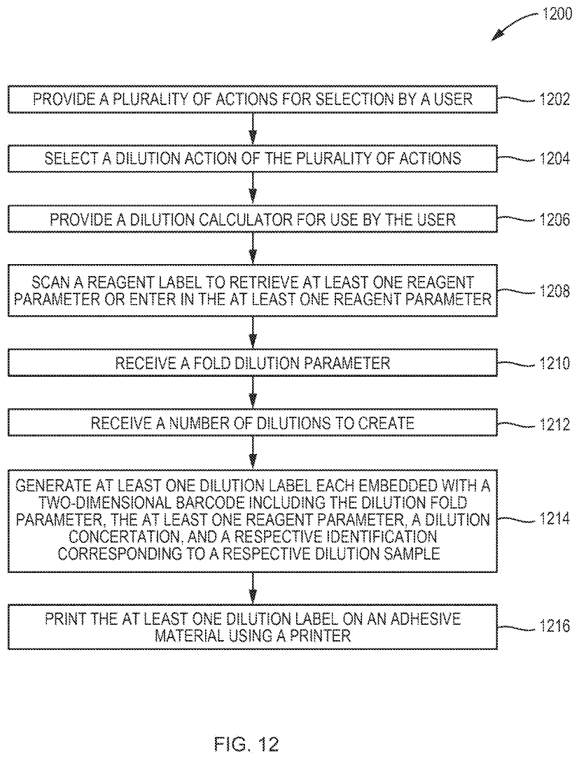

Referring to FIG. 3 and FIG. 12: a user may select the scan to dilute button 323 which can cause the computing device 200 initiate the method 1200, which illustrates a dilution in accordance with an embodiment of the present disclosure. According to an example, one or more process blocks of FIG. 12 may be performed by the computing device 200. A user may want to make serial dilutions of a reagent, drug, antibody, chemical, cell mixture, etc. The computing device can prompt the user to either enter the information (if the reagent does not have an existing barcode label) or to scan the barcode for the reagent that will be diluted. Upon scanning or entering, the computing device may ask the user to input the fold dilution (for example, a factor of 10) and the number of dilutions that they would like to create. In some embodiments, the computing device will automatically populate the correct number of labels, information of the reagent, the unique concentration for each dilution will be included as one of the text identifiers on the label. In some embodiments, a dilution calculator may be provided.

As shown in FIG. 12, the process 1200 may include providing a plurality of actions for selection by a user (block 1202). Act 1204 selects a dilution action of the plurality of actions (e.g., a user may press button 323 of FIG. 3). As is shown in FIG. 12, the process 1200 may include providing a dilution calculator for use by the user (block 1206). As further shown in FIG. 12, the process 1200 may include scanning a reagent label to retrieve at least one reagent parameter or enter in the at least one reagent parameter (block 1208). The process 1200 may include receiving a fold dilution parameter (block 1210). In Act 1212, the process 1200 may receive a number of dilutions to create, for example, as entered in by a user. As further shown in FIG. 12, the process 1200 may include generating at least one dilution label each embedded with a two-dimensional barcode including the dilution fold parameter, the at least one reagent parameter, a dilution concertation, and a respective identification corresponding to a respective dilution sample (block 1214). The computing device 200 may print one or more of the at least one dilution label on an adhesive material using a printer (block 1216).

It should be noted that while FIG. 12 shows example blocks of process 1200, in some implementations, the process 1200 may include additional blocks, fewer blocks, different blocks, or differently arranged blocks than those depicted in FIG. 12. Additionally, or alternatively, two or more of the blocks of process 1200 may be performed in parallel.

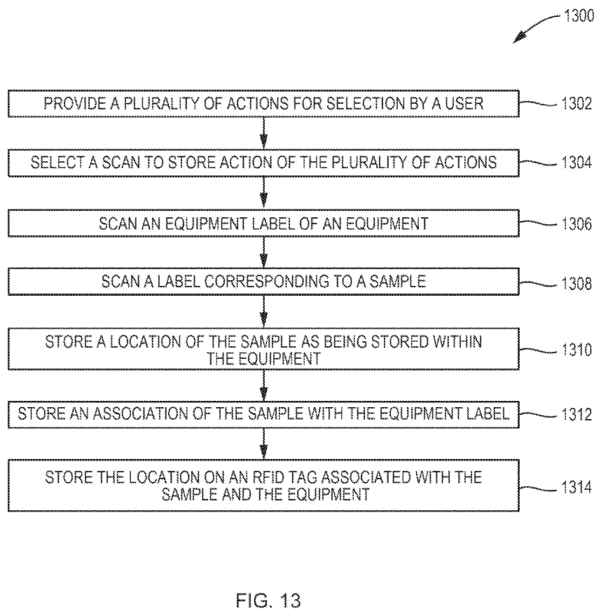

Referring to FIGS. 3 and 13, a user can select a scan to store button 324 to initiate a method 1300 shown in FIG. 13. That is, FIG. 13 shows a method 1300 that illustrates a scan-to-store action in accordance with an embodiment of the present disclosure. According to an example, one or more process blocks of FIG. 13 may be performed by the computing device 200.

In the laboratory setting, long-term or short-term storage of samples may be utilized. The computing device 200 may allow users to make an “equipment” label for equipment such as refrigerators, freezers or cryotanks. These equipment labels include information such as the name of the equipment, location of the equipment, last calibration date, number of shelves, temperature, etc. Upon choosing button 324, the user is prompted to scan the reagent that they wish to store first and then to scan the equipment label that indicates the location where the sample will be stored. Depending on the type of equipment scanned, users can add specific information (such as shelf number or strand number) and other notes that help to identify the sample when it needs to be retrieved. Upon “storing” a reagent in a specific location, the computing device 200 can add that reagent to a list compiled for the specific location/equipment label that is also searchable. Thus, if a sample is lost, users can search for the sample using keywords to find out where it is. In some embodiments, RFID-labels and an RFID-compatible barcode reader may utilized the supplement the inventory management of the samples, in order to more specifically identify physical location.

As shown in FIG. 13, the process 1300 may include providing a plurality of actions for selection by a user (block 1302). The user in Act 1304 may select a scan to store action of the plurality of actions and then in Act 1304 the use can scan an equipment label of an equipment. As further shown in FIG. 13, the process 1300 may include scanning a label corresponding to a sample (block 1308) and then the user in Act 1310 may store a location of the sample as being stored within the equipment (e.g., the information may be stored in a database). As also shown in FIG. 13, the process 1300 may include storing an association of the sample with the equipment label (block 1312). The Act 1312 stores the location on an RFID tag associated with the sample and the equipment.

It should be noted that while FIG. 13 shows example blocks of process 1300, in some implementations, the process 1300 may include additional blocks, fewer blocks, different blocks, or differently arranged blocks than those depicted in FIG. 13. Additionally, or alternatively, two or more of the blocks of process 1300 may be performed in parallel.

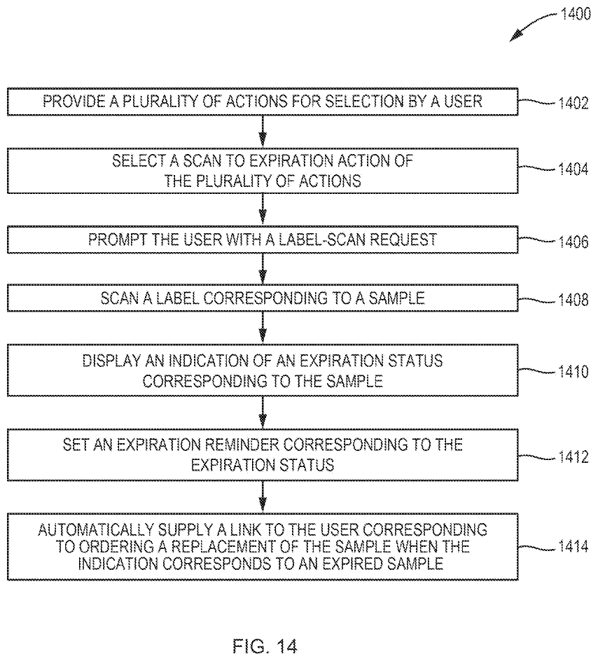

Referring now to FIGS. 3 and 14, a user can select button 326 to scan for expiration. That is, button 326 may initiate method 1400 of FIG. 14. FIG. 14 shows a flow chart diagram of a method 1400 to illustrate a scan-to-expiration action in accordance with an embodiment of the present disclosure. According to an example, one or more process blocks of FIG. 14 may be performed by the computing device 200.

Button 326 may be utilized at any stage of the workflow where the use of a potentially expired reagent could affect results in a negative way. The user can scan any label created previously and the computing device 200 can display an expiration date on the GUI. In the case of mixtures, the computing device 200 can display the closest expiration date to the current date but users can check expiration dates of every component if they wish. In some embodiments, users can also set reminders for reagents for in-app (or text, email, etc.) notifications when they are nearly expired. Users can define what “nearly” means in terms of time (for example, 2 days, 3 weeks, etc.).

In some additional embodiments, this feature can be automatic for every label created unless turned off by the user within their own profile settings, and the expiration date warning can be modified uniquely for every label prior to printing. Additionally, the user can click on the expiration date and the product ordering page of that reagent of interest that can appear on screen. For example, the computing device 200 may allow the user to specify an ordering link for reordering while setting up notifications manually.

As shown in FIG. 14, the process 1400 may include providing a plurality of actions for selection by a user (block 1402), which may be providing the button 326 for selection. A user can select a scan to expiration action of the plurality of actions by pressing the button 326 (block 1404). The computer device in Act 1408 may prompt the user with a label-scan request and the user can scan a label corresponding to a sample as Act 1410. The computing device 200 may display an indication of an expiration status corresponding to the sample (block 1410). As also shown in FIG. 14, the process 1400 may include setting an expiration reminder corresponding to the expiration status (block 1412) vis the computing device 200. The process 1400 may include automatically supplying a link to the user corresponding to ordering a replacement of the sample when the indication corresponds to an expired sample (block 1414).

It should be noted that while FIG. 14 shows example blocks of process 1400, in some implementations, the process 1400 may include additional blocks, fewer blocks, different blocks, or differently arranged blocks than those depicted in FIG. 14. Additionally, or alternatively, two or more of the blocks of process 1400 may be performed in parallel.

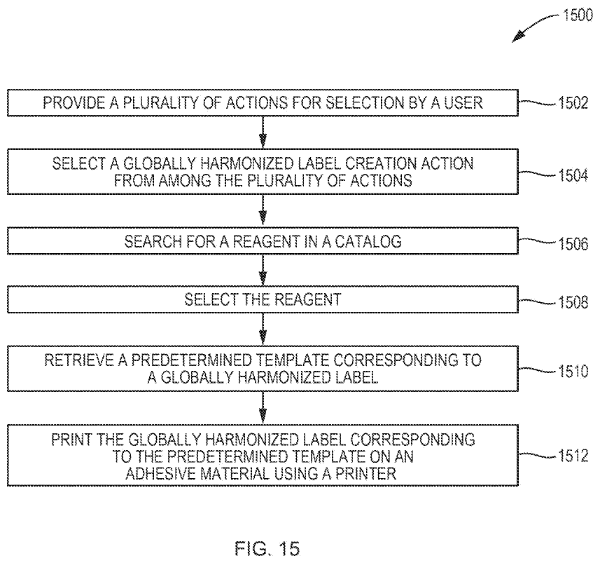

FIG. 15 shows a flow chart diagram of a method 1500 to illustrate a globally harmonized label creation action in accordance with an embodiment of the present disclosure. According to an example, one or more process blocks of FIG. 15 may be performed by the computing device 200.

The computing device 200 may assist in creating a GHS (Globally harmonized system) label for a chemical or reagent. In some embodiments, this option provides users with a textbox where they can search for a reagent in the a catalog. Additionally, they may scan an existing “scan me now” label to procure this information automatically. Then, the computing device can populate colored and pictorial labels for the reagent to print and apply onto aliquots, reagents, or experimental mixtures. Additionally, users can create custom GHS labels if they wish. In additional embodiments, scanning a barcode on GHS labels can provide users with a link to that product page and the Safety Data Sheet (SDS) for that particular chemical.

A user can scan to create a GHS label by pressing the button 330 which can initiate the process 1500 of FIG. 15. The process 1500 may include providing a plurality of actions for selection by a user (block 1502), such as by providing the button 330. A user can then select the button 330 to initiate a globally harmonized label creation action from among the plurality of actions (block 1504). The computing device 200 may then search for a reagent in a catalog (block 1506). As further shown in FIG. 15, the process 1500 may include selecting the reagent (block 1508). The computing device 200 may retrieve a predetermined template corresponding to a globally harmonized label (block 1510) and then print the globally harmonized label corresponding to the predetermined template on an adhesive material using a printer (block 1512).

It should be noted that while FIG. 15 shows example blocks of process 1500, in some implementations, the process 1500 may include additional blocks, fewer blocks, different blocks, or differently arranged blocks than those depicted in FIG. 15. Additionally, or alternatively, two or more of the blocks of process 1500 may be performed in parallel.

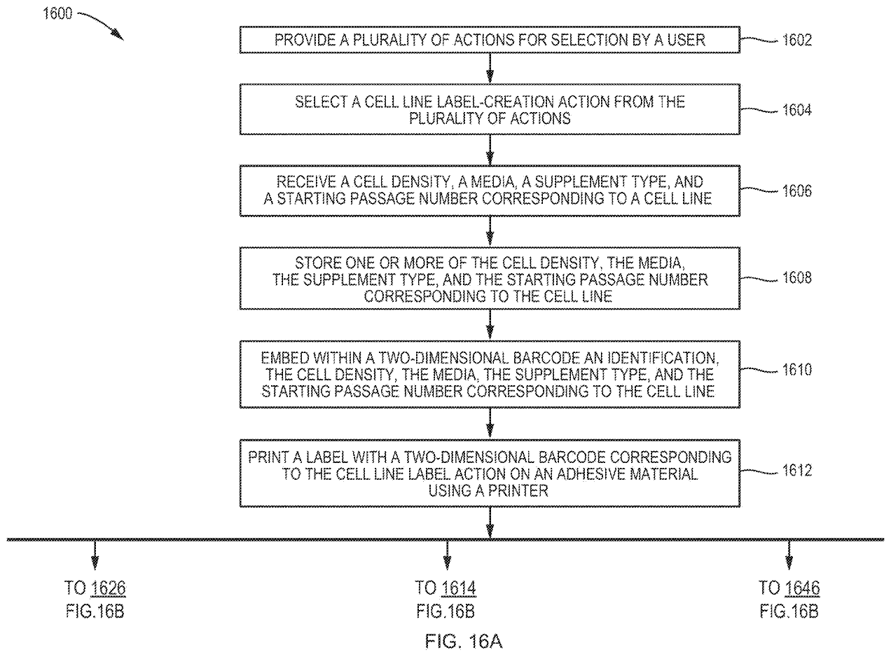

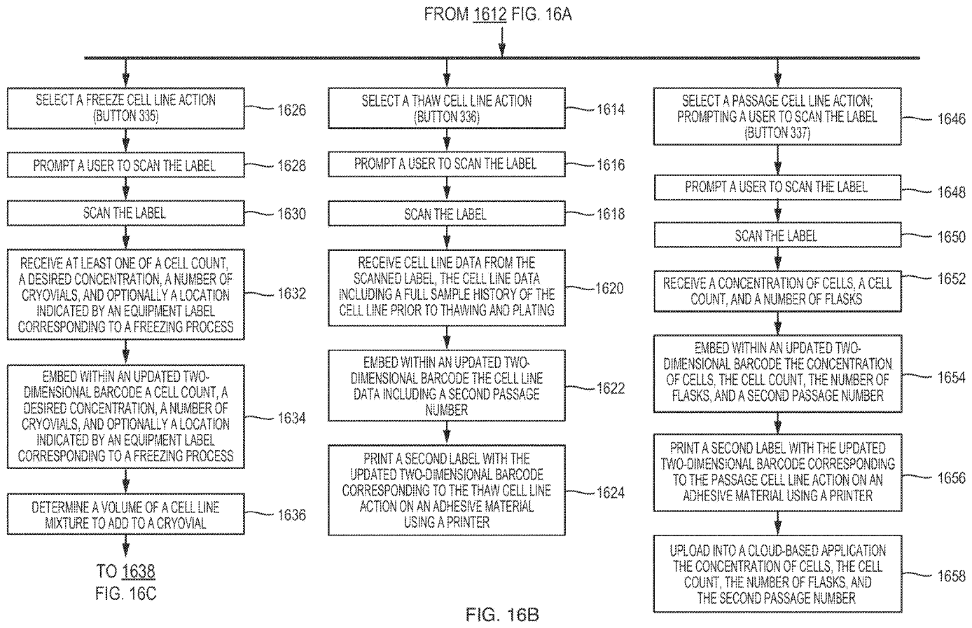

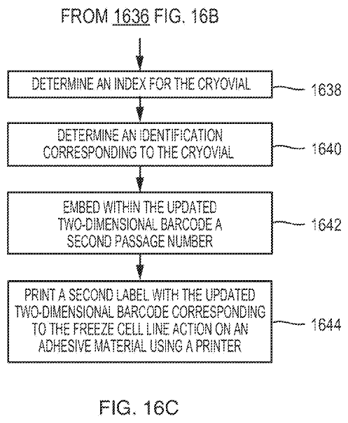

FIGS. 16A-16B shows a flow chart diagram of a method to illustrate cell line actions in accordance with an embodiment of the present disclosure. According to an example, one or more process blocks of FIG. 16 may be performed by the computing device 200. Initially a button 328 may be selected by a user. Thereafter, buttons 335-337 may become active as described below.

The computing device 200 may track thawing and freezing workflows in cell culture applications. The user may begin by designing and making a cell label for the cell lines that they wish to culture, which includes aspects such as cell density, media and supplement type and starting passage number. Upon selecting on one of these options below, the user can automatically transfer this information and populate new labels for one of the following workflows that correspond to buttons 335-337.

As shown in FIG. 16A, the process 1600 may include providing a plurality of actions for selection by a user (block 1602). The process 1600 may include selecting a cell line label-creation action (Button 328) from the plurality of actions (block 1604). Act 1606 receives a cell density, a media, a supplement type, and a starting passage number corresponding to a cell line. This information may be stored, e.g., in a database in storing one or more of the cell density, the media, the supplement type, and the starting passage number corresponding to the cell line (block 1608). As in addition shown in FIG. 16, the process 1600 may include embedding within a two-dimensional barcode an identification, the cell density, the media, the supplement type, and the starting passage number corresponding to the cell line (block 1610). The computing device in Act 1612 may print a label with a two-dimensional barcode corresponding to the cell line label action on an adhesive material using a printer. Referring now to FIG. 16B, a user may then select one of buttons 335-337.

Button 335 selects a freeze cell line action 1614. Upon selecting, the user is able to scan their cell label and the computing device 200 will generate new labels automatically for the cell line with the existing passage number on it, in some embodiments. Upon scanning the cell label, the computing device can prompt the user to gather information about cell count, desired concentration and number of cryovials that will be frozen down. If desired, the user can link this information to the location (equipment label) where the cryovials will be stored. In yet additional embodiments, the computing device 200 can, based off of cell count, calculate how much volume of their cell mixture the user will need to add to each cryovial. The computing device 200 may also index the full sample history of the cells during their time of culture for recordkeeping purposes. The computing device 200 may include a built-in timer with the ability for notifications, pre-programmed by the user for the freezing process.

Act 1626 selects a freeze cell line action (e.g., by a user pressing the button 335). Act 1628 prompts a user to scan the label. Act 1630 scans the label. Act 1632 receives at least one of a cell count, a desired concentration, a number of cryovials, and optionally a location indicated by an equipment label corresponding to a freezing process. Act 1634 embeds within an updated two-dimensional barcode a cell count, a desired concentration, a number of cryovials, and optionally a location indicated by an equipment label corresponding to a freezing process. Act 1636 determines a volume of a cell line mixture to add to a cryovial. Act 1638 determines an index for the cryovial. Act 1640 determines an identification corresponding to the cryovial. Act 1642 embeds within the updated two-dimensional barcode a second passage number. Act 1644 prints a second label with the updated two-dimensional barcode corresponding to the freeze cell line action on an adhesive material using a printer.

A user may also select a thaw cell line action by pressing button 336 in Act 1614. Cell labels may be applied to those cells that have been frozen or have arrived in the lab for the first time may be scanned within this workflow. The computing device 200, in some embodiments, will automatically populate a new cell label for that cell line based off the information from the barcode and automatically advance the passage number of the cells. Users may also read information about the media requirements for that cell line after scanning and see a full sample history of this cell line prior to thawing and plating.

Act 1614 selects a thaw cell line action. Act 1616 prompts a user to scan the label. Act 1618 scans the label. Act 1620 receives cell line data from the scanned label. The cell line data may include a full sample history of the cell line prior to thawing and plating. Act 1622 embeds within an updated two-dimensional barcode the cell line data including a second passage number. Act 1624 prints a second label with the updated two-dimensional barcode corresponding to the thaw cell line action on an adhesive material using a printer.

The process 1600 further includes selecting a passage cell line action in Act 1646, e.g., by pressing the button 337. Upon clicking this option, the computing device can prompt the user to scan the existing barcode on the cell label for the cell line they wish to passage. Once scanned, the computing device may ask the user to input the concentration of cells desired, the cell count, and the number of flasks desired, and will use it to automatically generate a new label for the new cell line and new passage number with the existing information. In yet additional embodiments, all cell culture information may be uploaded into a cloud-based application where data is saved side-by-side with images of the sample, Scepter histograms, TEER information or other types of analytical data.

Act 1648 prompts a user to scan the label. A user in Act 1650 can scan the label. Act 1652 receives a concentration of cells, a cell count, and a number of flasks. Act 1654 embeds within an updated two-dimensional barcode the concentration of cells, the cell count, the number of flasks, and a second passage number. Act 1656 prints a second label with the updated two-dimensional barcode corresponding to the passage cell line action on an adhesive material using a printer. Act 1658 uploads into a cloud-based application the concentration of cells, the cell count, the number of flasks, and the second passage number.

It should be noted that while FIG. 16 shows example blocks of process 1600, in some implementations, the process 1600 may include additional blocks, fewer blocks, different blocks, or differently arranged blocks than those depicted in FIG. 16. Additionally, or alternatively, two or more of the blocks of process 1600 may be performed in parallel.

Referring again to FIG. 3, a user may select the Button 312 to update a sample history label. Thereafter, a GUI with all of the information related to the sample history may be presented to the user. The user can modify all/any of the data in the database and/or may print out a new label embedded the new information in a new label.

Each of the characteristics and examples described herein, and combinations thereof, may be said to be encompassed by embodiments described herein. The present disclosure therefore describes the following non-limiting aspects, various, or combinations thereof:
(1) A method for guiding a worker in laboratory workflow handling, the method comprising: providing a plurality of actions for selection by a user; selecting an action of the plurality of actions; determining an identification corresponding to a sample; embedding within a two-dimensional barcode the identification and workflow data corresponding to the sample; and printing a label with the two-dimensional barcode on an adhesive material using a printer.
(2) The method according to aspect 1, wherein the workflow data is a string in human-readable format.
(3) The method according to aspect 2, wherein the workflow data is experimental utilization of the sample.
(4) The method according to aspect 1, further comprising a guided menu configured to guide the user to input the workflow data.
(5) The method according to aspect 1, wherein the selected action is an aliquot action.
(6) The method according to aspect 5, further comprising: providing a create reagent label action; and providing a create aliquot labels action.
(7) The method according to aspect 6, further comprising: selecting the create reagent label action; prompting the user with a request for a plurality of reagent parameters; receiving the plurality of reagent parameters; and embedding within the two-dimensional barcode the plurality of reagent parameters corresponding to the sample.
(8) The method according to aspect 7, further comprising storing the plurality of reagent parameters and the identification corresponding to the sample in a database.
(9) The method according to aspect 7, further comprising: selecting an option to save label data corresponding to the two-dimensional barcode; and saving the label data in a selected one of a plurality of file formats.
(10) The method according to aspect 7, wherein the plurality of reagent parameters includes at least one of a product name, a concentration, a formulation, a total volume, an expiration, a catalog number, a vendor, a lot number, a batch number, and a note.
(11) The method according to aspect 7, further comprising selecting a label format from a plurality of label formats, wherein the act of printing the label includes printing the label to correspond with the label format.
(12) The method according to aspect 11, further comprising: selecting a scan barcode action; prompting the user with a label-scan request; scanning a barcode of a label; and selecting an option to save label data corresponding to the two-dimensional barcode.
(13) The method according to aspect 12, further comprising saving the label data in a selected one of a plurality of file formats.
(14) The method according to aspect 7, further comprising storing the plurality of reagent parameters as a template in a datastore.
(15) The method according to aspect 10, further comprising storing the identification in a datastore and associated with a template.
(16) The method according to aspect 7, further comprising providing a text string configured to be readable on the label adjacent to the two-dimensional barcode, wherein the act of printing the label includes printing the text string adjacent to the two-dimensional barcode.
(17) The method according to aspect 6, further comprising: selecting the create aliquot labels action; prompting the user with a label-scan request; scanning a preexisting label of a reagent in response to the label-scan request; identifying the sample based upon the identification embedded within the preexisting label; retrieving a plurality of reagent parameters corresponding to the sample; prompting the user with a request for at least one aliquot parameter including a number of aliquots; receiving the at least one aliquot parameter including the number of aliquots; embedding within the two-dimensional barcode the at least one aliquot parameter; adding a second text string configured to be readable on the label adjacent to the two-dimensional barcode; and printing a plurality of aliquot labels corresponding to the number of aliquots, wherein the plurality of labels includes the label.
(18) The method according to aspect 17, further comprising storing the plurality of reagent parameters, wherein the identification corresponds to the sample and the at least one aliquot parameter.
(19) The method according to aspect 18, wherein the at least on aliquot parameter is a volume of fluid remaining, the method comprising notifying the user if the volume of fluid remaining is below a threshold for a predetermined number of aliquots.
(20) The method according to aspect 18, further comprising: storing the number of aliquots in a database; storing a number of aliquots remaining in the database; and notify the user if the number of aliquots remaining is below a predetermined threshold.
(21) The method according to aspect 20, further comprising directing the user to a product page link in response to the number of aliquots remaining below a predetermined threshold.
(22) The method according to aspect 20, further comprising automatically re-ordering the reagent in response to the number of aliquots remaining below a predetermined threshold.
(23) The method according to aspect 17, further comprising: selecting an option to save label data corresponding to the two-dimensional barcode; and saving the label data in a selected one of a plurality of file formats.
(24) The method according to aspect 17, further comprising: retrieving a plurality of reagent parameters corresponding to the preexisting label of the reagent; and embedding within the two-dimensional barcode the plurality of reagent parameters.
(25) The method according to aspect 24, further comprising: selecting a modify two-dimensional barcode action; providing the plurality of reagent parameters; and modifying at least one of the plurality of reagent parameters, wherein the plurality of aliquot labels are printed in accordance with the modified at least one of the plurality of reagent parameters.
(26) The method according to aspect 25, further comprising: selecting an option to save label data corresponding to the two-dimensional barcode; and saving the label data in a selected one of a plurality of file formats.
(27) The method according to aspect 17, further comprising: selecting a scan barcode action; prompting the user with a label-scan request; scanning at least one of the plurality of aliquot labels; selecting a deduct action; prompting the user with a deduct amount and a number deducted of aliquots; and deducting the deduct amount from the number of aliquots.
(28) The method according to aspect 17, further comprising printing an updated plurality of aliquot labels corresponding to the number of deduced aliquots, wherein each of the updated plurality of aliquot labels embeds an updated aliquot volume in a respective two-dimensional barcode on a respective one of the updated plurality of aliquot labels.
(29) The method according to aspect 28, further comprising: selecting an option to save label data corresponding to the two-dimensional barcode; and saving the label data in a selected one of a plurality of file formats.
(30) The method according to aspect 1, wherein the selected action is a sample history action.
(31) The method according to aspect 30, further comprising: providing a create new sample-history label action; and providing an update sample history label action.
(32) The method according to aspect 31, further comprising: selecting the create new sample-history label action; providing a sample history label format; adding a first text string configured for embedding within the two-dimensional barcode; adding a second text string configured to be readable on the label adjacent to the two-dimensional barcode; and printing the label with the two-dimensional barcode on an adhesive material using a printer.
(33) The method according to aspect 32, further comprising adding a timestamp to one of the first text string and the second text string corresponding to a create time of the label.
(34) The method according to aspect 32, further comprising: selecting an option to save label data corresponding to the two-dimensional barcode; and saving the label data in a selected one of a plurality of file formats.
(35) The method according to aspect 31, further comprising: selecting the create new sample-history label action; providing a sample history label format; adding a text string associated with the identification; and storing the text string associated with the identification in a database.
(36) The method according to aspect 31, further comprising: selecting the update sample history label action; updating a text string associated with the identification; and storing the updated text string associated with the identification in a database.
(37) The method according to aspect 31, further comprising: selecting the update sample history label action; prompting the user with a label-scan request; scanning a preexisting label in response to the label-scan request; identifying the sample based upon the identification embedded within the preexisting label; adding a second text string for appending to a first text string, wherein the first and second text string are configured for embedding within the two-dimensional barcode; and adding a third text string configured to be readable on the label adjacent to the two-dimensional barcode.
(38) The method according to aspect 37, further comprising adding a timestamp to one of the second text string and the third text string corresponding to an update time of the label.
(39) The method according to aspect 37, further comprising: selecting an option to save label data corresponding to the two-dimensional barcode; and saving the label data in a selected one of a plurality of file formats.
(40) The method according to aspect 1, further comprising scanning a label, wherein an action for selection of the plurality of actions for selection by the user is an action to duplicate the scanned label, wherein the label is at least one duplicated label.
(41) The method according to aspect 40, further comprising: displaying at least one parameter corresponding to the scanned label; and prompting the user for modification of the at least one parameter, wherein the at least one duplicated label is printed in accordance with the modified at least one parameter.
(42) The method according to aspect 1, further comprising: scanning a first label of the sample; scanning a second label of another sample; selecting a mixture action; receiving a plurality of parameters corresponding to the mixture action; and embedding within the two-dimensional barcode the plurality of parameters corresponding to the sample.
(43) The method according to aspect 42, further comprising automatically generating at least one of the plurality of parameters corresponding to the mixture action.
(44) The method according to aspect 42, further comprising saving a laboratory recipe corresponding to the plurality of parameters corresponding to the mixture action.
(45) The method according to aspect 42, further comprising determining an amount of a particular reagent should be added into a mixture based upon the mixture action to thereby achieve a predetermined concentration.
(46) The method according to aspect 42, further comprising embedding a plate map within the two-dimensional barcode.
(47) The method according to aspect 1, further comprising: scanning a label, wherein an action for selection of the plurality of actions for selection by the user is an action to dilute the sample corresponding to the scanned label; receiving a dilution amount; and embedding within the two-dimensional barcode the dilution amount corresponding to the sample.
(48) The method according to aspect 1, further comprising: scanning an equipment label; scanning a label corresponding to the sample; and storing a location of the sample as being stored within an equipment associated with the equipment label.
(49) The method according to aspect 48, further comprising storing the location on an RFID tag associated with at least one of the sample and the equipment.
(50) The method according to aspect 1, further comprising: scanning an equipment label; scanning a label corresponding to the sample; and storing an association of the sample with an equipment associated with the equipment label.
(51) The method according to aspect 1, further comprising: scanning a label corresponding to the sample; and displaying an indication of an expiration status corresponding to the sample.
(52) The method according to aspect 51, further comprising automatically supplying a link to the user corresponding to ordering a replacement of the sample when the indication corresponds to an expired sample.
(53) The method according to aspect 52, further comprising setting an expiration reminder corresponding to the expiration status.
(54) The method according to aspect 1, further comprising automatically setting an expiration reminder corresponding to the sample.
(55) The method according to aspect 1, further comprising: selecting a globally harmonized label creation action; searching for a reagent in a catalog; selecting the reagent; and retrieving a predetermined label format corresponding to the reagent, wherein the two-dimensional barcode corresponds to the predetermined label format.
(56) The method according to aspect 1, further comprising: scanning a label; and retrieving a predetermined template corresponding to the label, wherein the predetermined template corresponds to a globally harmonized label, wherein the two-dimensional barcode corresponds to the predetermined template.
(57) The method according to aspect 1, wherein the sample is a cell line, the method further comprising selecting a cell line action, wherein the two-dimensional barcode corresponds to the cell line action.
(58) The method according to aspect 57, wherein the cell line action is one of freezing, thawing, and storing the cell line.
(59) The method according to aspect 57, further comprising storing at least one of a cell density, a media, a supplement type, and a starting passage number corresponding to the cell line.
(60) The method according to aspect 57, further comprising receiving at least one of a cell count, a desired concentration, and a number of cryovials for embedding within the two-dimensional barcode.
(61) The method according to aspect 57, further comprising determining a volume of a cell line mixture to add to a cryovial; and determining an index for the cryovial.
(62) The method according to aspect 1, further comprising: receiving at least one reagent parameter; receiving a label format; generate a template corresponding to the label format the at least one reagent parameter; and saving the template for reuse.
(63) The method according to aspect 1, wherein the identification is a unique identification.
(64) The method according to aspect 1, wherein the two-dimensional barcode encodes the sample workflow data in a standardized two-dimensional barcode format.
(65) The method according to aspect 1, further comprising selecting a label format from among a plurality of label formats, wherein each label format of the plurality of label formats is configured to attached to respective container type.
(66) The method according to aspect 1, wherein the method is implemented on a computer using an operative set of processor executable instructions configured to cause the computer to realize the method.
(67) A device for guiding a worker in laboratory workflow handling, the device comprising: one or more processors configured to: provide a plurality of actions for selection by a user; select an action of the plurality of actions; determine an identification corresponding to a sample; embed within a two-dimensional barcode the identification and workflow data corresponding to the sample; and print a label with the two-dimensional barcode on an adhesive material using a printer.
(68) The device of aspect 67, wherein the workflow data is a string in human-readable format.
(69) The device of aspect 68, wherein the workflow data is experimental utilization of the sample.
(70) The device of aspect 67, wherein the one or more processors are further configured to a guided menu configured to guide the user to input the workflow data.
(71) The device of aspect 67, wherein the selected action is an aliquot action.
(72) The device of aspect 71, wherein the one or more processors are further configured to: provide a create reagent label action; and provide a create aliquot labels action.
(73) The device of aspect 72, wherein the one or more processors are further configured to: select the create reagent label action; prompt the user with a request for a plurality of reagent parameters; receive the plurality of reagent parameters; and embed within the two-dimensional barcode the plurality of reagent parameters corresponding to the sample.
(74) The device of aspect 73, wherein the one or more processors are further configured to store the plurality of reagent parameters and the identification corresponding to the sample in a database.
(75) The device of aspect 73, wherein the one or more processors are further configured to: select an option to save label data corresponding to the two-dimensional barcode; and save the label data in a selected one of a plurality of file formats.
(76) The device of aspect 73, wherein the plurality of reagent parameters includes at least one of a product name, a concentration, a formulation, a total volume, an expiration, a catalog number, a vendor, a lot number, a batch number, and a note.
(77) The device of aspect 76, wherein the one or more processors are further configured to store the identification in a datastore and associated with a template.
(78) The device of aspect 73, wherein the one or more processors are further configured to select a label format from a plurality of label formats, wherein the act of printing the label includes printing the label to correspond with the label format.
(79) The device of aspect 78, wherein the one or more processors are further configured to: select a scan barcode action; prompt the user with a label-scan request; scan a barcode of a label; and select an option to save label data corresponding to the two-dimensional barcode.
(80) The device of aspect 79, wherein the one or more processors are further configured to save the label data in a selected one of a plurality of file formats.
(81) The device of aspect 73, wherein the one or more processors are further configured to store the plurality of reagent parameters as a template in a datastore.
(82) The device of aspect 73, wherein the one or more processors are further configured to provide a text string configured to be readable on the label adjacent to the two-dimensional barcode, wherein the act of printing the label includes printing the text string adjacent to the two-dimensional barcode.
(83) The device of aspect 72, wherein the one or more processors are further configured to: select the create aliquot labels action; prompt the user with a label-scan request; scan a preexisting label of a reagent in response to the label-scan request; identify the sample based upon the identification embedded within the preexisting label; retrieve a plurality of reagent parameters corresponding to the sample; prompt the user with a request for at least one aliquot parameter including a number of aliquots; receive the at least one aliquot parameter including the number of aliquots; embed within the two-dimensional barcode the at least one aliquot parameter; add a second text string configured to be readable on the label adjacent to the two-dimensional barcode; and print a plurality of aliquot labels corresponding to the number of aliquots, wherein the plurality of labels includes the label.
(84) The device of aspect 83, wherein the one or more processors are further configured to store the plurality of reagent parameters, wherein the identification corresponds to the sample and the at least one aliquot parameter.
(85) The device of aspect 84, wherein the one or more processors, when the at least on aliquot parameter is a volume of fluid remaining, the method, are configured to notify the user if the volume of fluid remaining is below a threshold for a predetermined number of aliquots.
(86) The device of aspect 84, wherein the one or more processors are further configured to: store the number of aliquots in a database; store a number of aliquots remaining in the database; and notify the user if the number of aliquots remain is below a predetermined threshold.
(87) The device of aspect 86, wherein the one or more processors are further configured to direct the user to a product page link in response to the number of aliquots remaining below a predetermined threshold.
(88) The device of aspect 86, wherein the one or more processors are further configured to automatically re-order the reagent in response to the number of aliquots remaining below a predetermined threshold.
(89) The device of aspect 83, wherein the one or more processors are further configured to: select an option to save label data corresponding to the two-dimensional barcode; and save the label data in a selected one of a plurality of file formats.
(90) The device of aspect 83, wherein the one or more processors are further configured to: retrieve a plurality of reagent parameters corresponding to the preexisting label of the reagent; and embed within the two-dimensional barcode the plurality of reagent parameters.
(91) The device of aspect 90, wherein the one or more processors are further configured to: select a modify two-dimensional barcode action; provide the plurality of reagent parameters; and modify at least one of the plurality of reagent parameters, wherein the plurality of aliquot labels are printed in accordance with the modified at least one of the plurality of reagent parameters.
(92) The device of aspect 91, wherein the one or more processors are further configured to: select an option to save label data corresponding to the two-dimensional barcode; and save the label data in a selected one of a plurality of file formats.
(93) The device of aspect 83, wherein the one or more processors are further configured to: select a scan barcode action; prompt the user with a label-scan request; scan at least one of the plurality of aliquot labels; select a deduct action; prompt the user with a deduct amount and a number deducted of aliquots; and deduct the deduct amount from the number of aliquots.
(94) The device of aspect 83, wherein the one or more processors are further configured to print an updated plurality of aliquot labels corresponding to the number of deduced aliquots, wherein each of the updated plurality of aliquot labels embeds an updated aliquot volume in a respective two-dimensional barcode on a respective one of the updated plurality of aliquot labels.
(95) The device of aspect 94, wherein the one or more processors are further configured to: select an option to save label data corresponding to the two-dimensional barcode; and save the label data in a selected one of a plurality of file formats.
(96) The device of aspect 67, wherein the selected action is a sample history action.
(97) The device of aspect 96, wherein the one or more processors are further configured to: provide a create new sample-history label action; and provide an update sample history label action.
(98) The device of aspect 97, wherein the one or more processors are further configured to: select the create new sample-history label action; provide a sample history label format; add a first text string configured for embedding within the two-dimensional barcode; add a second text string configured to be readable on the label adjacent to the two-dimensional barcode; and print the label with the two-dimensional barcode on an adhesive material using a printer.
(99) The device of aspect 98, wherein the one or more processors are further configured to add a timestamp to one of the first text string and the second text string corresponding to a create time of the label.
(100) The device of aspect 98, wherein the one or more processors are further configured to: select an option to save label data corresponding to the two-dimensional barcode; and save the label data in a selected one of a plurality of file formats.
(101) The device of aspect 97, wherein the one or more processors are further configured to: select the create new sample-history label action; provide a sample history label format; add a text string associated with the identification; and store the text string associated with the identification in a database.
(102) The device of aspect 97, wherein the one or more processors are further configured to: select the update sample history label action; update a text string associated with the identification; and store the updated text string associated with the identification in a database.
(103) The device of aspect 97, wherein the one or more processors are further configured to: select the update sample history label action; prompt the user with a label-scan request; scan a preexisting label in response to the label-scan request; identify the sample based upon the identification embedded within the preexisting label; add a second text string for appending to a first text string, wherein the first and second text string are configured for embedding within the two-dimensional barcode; and add a third text string configured to be readable on the label adjacent to the two-dimensional barcode.
(104) The device of aspect 103, wherein the one or more processors are further configured to add a timestamp to one of the second text string and the third text string corresponding to an update time of the label.
(105) The device of aspect 103, wherein the one or more processors are further configured to: select an option to save label data corresponding to the two-dimensional barcode; and save the label data in a selected one of a plurality of file formats.
(106) The device of aspect 67, wherein the one or more processors are further configured to scan a label, wherein an action for selection of the plurality of actions for selection by the user is an action to duplicate the scanned label, wherein the label is at least one duplicated label.
(107) The device of aspect 106, wherein the one or more processors are further configured to: display at least one parameter corresponding to the scanned label; and prompt the user for modification of the at least one parameter, wherein the at least one duplicated label is printed in accordance with the modified at least one parameter.
(108) The device of aspect 67, wherein the one or more processors are further configured to: scan a first label of the sample; scan a second label of another sample; select a mixture action; receive a plurality of parameters corresponding to the mixture action; and embed within the two-dimensional barcode the plurality of parameters corresponding to the sample.
(109) The device of aspect 108, wherein the one or more processors are further configured to automatically generate at least one of the plurality of parameters corresponding to the mixture action.
(110) The device of aspect 108, wherein the one or more processors are further configured to save a laboratory recipe corresponding to the plurality of parameters corresponding to the mixture action.
(111) The device of aspect 108, wherein the one or more processors are further configured to determine an amount of a particular reagent should be added into a mixture based upon the mixture action to thereby achieve a predetermined concentration.
(112) The device of aspect 108, wherein the one or more processors are further configured to embed a plate map within the two-dimensional barcode.
(113) The device of aspect 67, wherein the one or more processors are further configured to: scan a label, wherein an action for selection of the plurality of actions for selection by the user is an action to dilute the sample corresponding to the scanned label; receive a dilution amount; and embed within the two-dimensional barcode the dilution amount corresponding to the sample.
(114) The device of aspect 67, wherein the one or more processors are further configured to: scan an equipment label; scan a label corresponding to the sample; and store a location of the sample as being stored within an equipment associated with the equipment label.
(115) The device of aspect 114, wherein the one or more processors are further configured to store the location on an RFID tag associated with at least one of the sample and the equipment.
(116) The device of aspect 67, wherein the one or more processors are further configured to: scan an equipment label; scan a label corresponding to the sample; and store an association of the sample with an equipment associated with the equipment label.
(117) The device of aspect 67, wherein the one or more processors are further configured to: scan a label corresponding to the sample; and display an indication of an expiration status corresponding to the sample.
(118) The device of aspect 117, wherein the one or more processors are further configured to automatically supply a link to the user corresponding to ordering a replacement of the sample when the indication corresponds to an expired sample.
(119) The device of aspect 118, wherein the one or more processors are further configured to set an expiration reminder corresponding to the expiration status.
(120) The device of aspect 67, wherein the one or more processors are further configured to automatically set an expiration reminder corresponding to the sample.
(121) The device of aspect 67, wherein the one or more processors are further configured to: select a globally harmonized label creation action; search for a reagent in a catalog; select the reagent; retrieve a predetermined label format corresponding to the reagent; and the two-dimensional barcode corresponds to the predetermined label format.
(122) The device of aspect 67, wherein the one or more processors are further configured to: scan a label; and retrieve a predetermined template corresponding to the label, wherein the predetermined template corresponds to a globally harmonized label, wherein the two-dimensional barcode corresponds to the predetermined template.
(123) The device of aspect 67, wherein the sample is a cell line, the method further comprising selecting a cell line action, wherein the two-dimensional barcode corresponds to the cell line action.
(124) The device of aspect 123, wherein the cell line action is one of freezing, thawing, and storing the cell line.
(125) The device of aspect 123, wherein the one or more processors are further configured to store at least one of a cell density, a media, a supplement type, and a starting passage number corresponding to the cell line.
(126) The device of aspect 123, wherein the one or more processors are further configured to receive at least one of a cell count, a desired concentration, and a number of cryovials for embedding within the two-dimensional barcode.
(127) The device of aspect 123, wherein the one or more processors are further configured to: determine a volume of a cell line mixture to add to a cryovial; and determine an index for the cryovial.
(128) The device of aspect 67, wherein the one or more processors are further configured to: receive at least one reagent parameter; receive a label format; generate a template correspond to the label format the at least one reagent parameter; and save the template for reuse.
(129) The device of aspect 67, wherein the identification is a unique identification.
(130) The device of aspect 67, wherein the two-dimensional barcode encodes the sample workflow data in a standardized two-dimensional barcode format.
(131) The device of aspect 67, wherein the one or more processors are further configured to select a label format from among a plurality of label formats, wherein each label format of the plurality of label formats is configured to attached to respective container type.
(132) The device of aspect 67, wherein the method is implemented on a computer using an operative set of processor executable instructions configured to cause the computer to realize the method.
(133) The device according to aspect 67, wherein the device is one of a computer, a smartphone, a PDA, a printer, a webserver, a server, a cloud server, a stand-alone printer, a handheld printer, a laptop, a wearable computer, and an embedded device.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in the drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g., “a,” “an,” or “the,” this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B. This expression signifies that, with respect to the present disclosure, the only relevant components of the device are A and B.

Furthermore, the terms “first,” “second,” “third,” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

What is claimed is:

1. A method for guiding a worker in laboratory workflow handling, the method comprising:
providing a plurality of actions for selection by a user;
selecting an action of the plurality of actions;
determining an identification corresponding to a sample;
embedding within a two-dimensional barcode the identification and workflow data corresponding to the sample; and
printing a label with the two-dimensional barcode on an adhesive material using a printer.

2. The method according to claim 1, wherein the workflow data is a string in human-readable format.

3. The method according to claim 2, wherein the workflow data is experimental utilization of the sample.

4. The method according to claim 1, further comprising a guided menu configured to guide the user to input the workflow data.

5. The method according to claim 1, wherein the selected action is an aliquot action.

6. The method according to claim 5, further comprising:
providing a create reagent label action; and
providing a create aliquot labels action.

7. The method according to claim 6, further comprising:
selecting the create reagent label action;
prompting the user with a request for a plurality of reagent parameters;
receiving the plurality of reagent parameters; and
embedding within the two-dimensional barcode the plurality of reagent parameters corresponding to the sample.

8. The method according to claim 7, further comprising storing the plurality of reagent parameters and the identification corresponding to the sample in a database.

9. The method according to claim 7, further comprising:
selecting an option to save label data corresponding to the two-dimensional barcode; and
saving the label data in a selected one of a plurality of file formats.

10. The method according to claim 7, wherein the plurality of reagent parameters includes at least one of a product name, a concentration, a formulation, a total volume, an expiration, a catalog number, a vendor, a lot number, a batch number, and a note.

11. The method according to claim 7, further comprising selecting a label format from a plurality of label formats, wherein the act of printing the label includes printing the label to correspond with the label format.

12. The method according to claim 11, further comprising:
selecting a scan barcode action;
prompting the user with a label-scan request;
scanning a barcode of a label; and
selecting an option to save label data corresponding to the two-dimensional barcode.

13. The method according to claim 12, further comprising saving the label data in a selected one of a plurality of file formats.

14. The method according to claim 7, further comprising storing the plurality of reagent parameters as a template in a datastore.

15. The method according to claim 10, further comprising storing the identification in a datastore and associated with a template.

16. The method according to claim 7, further comprising providing a text string configured to be readable on the label adjacent to the two-dimensional barcode, wherein the act of printing the label includes printing the text string adjacent to the two-dimensional barcode.

17. The method according to claim 6, further comprising:
selecting the create aliquot labels action;
prompting the user with a label-scan request;
scanning a preexisting label of a reagent in response to the label-scan request;
identifying the sample based upon the identification embedded within the preexisting label;
retrieving a plurality of reagent parameters corresponding to the sample;
prompting the user with a request for at least one aliquot parameter including a number of aliquots;
receiving the at least one aliquot parameter including the number of aliquots;
embedding within the two-dimensional barcode the at least one aliquot parameter;
adding a second text string configured to be readable on the label adjacent to the two-dimensional barcode; and
printing a plurality of aliquot labels corresponding to the number of aliquots, wherein the plurality of labels includes the label.

18. The method according to claim 17, further comprising storing the plurality of reagent parameters, wherein the identification corresponds to the sample and the at least one aliquot parameter.

19. The method according to claim 18, wherein the at least on aliquot parameter is a volume of fluid remaining, the method comprising notifying the user if the volume of fluid remaining is below a threshold for a predetermined number of aliquots.

20. The method according to claim 18, further comprising:
storing the number of aliquots in a database;
storing a number of aliquots remaining in the database; and
notify the user if the number of aliquots remaining is below a predetermined threshold.
